(19) United States
(12) Patent Application Publication
(10) Pub. No.: US 2026/0245685 A1
(43) Pub. Date: Aug. 20, 2026

(54) CLINICAL TRIAL EMULATION USING MULTI-AGENT LEARNING

(71) Applicant: Cornell University, Ithaca, NY (US)

(72) Inventors: Fei Wang, Chappaqua, NY (US); Weishen Pan, Little Neck, NY (US); Haoyang Li, New York, NY (US); Suraj Rajendran, New York, NY (US)

(21) Appl. No.: 19/540,357

(22) Filed: Feb. 13, 2026

Related U.S. Application Data
(60) Provisional application No. 63/758,778, filed on Feb. 14, 2025.
(60) Provisional application No. 63/783,861, filed on Apr. 4, 2025.

(51) Int. Cl. G16H 10/60; G16H 15/00

(52) U.S. Cl. CPC G16H 10/60; G16H 15/00

(57) ABSTRACT

Aspects of the present disclosure involve a system comprising a computer-readable storage medium storing a program and method for clinical trial emulation using multi-agent learning. The program and method provide for receiving a target trial specification comprising eligibility criteria, treatments, and outcomes; extracting key components and temporal information using a large language model and generating structured target trial definitions compatible with standardized clinical data formats; generating executable database queries and retrieving patient clinical data from an electronic health record database to produce an analysis-ready dataset; performing covariate balancing and survival analysis using causal inference methods to estimate treatment effects; and iteratively refining the target trial definitions and treatment effect estimates by communicating feedback via a multi-agent system comprising neural network models optimized through reinforcement learning with human feedback to generate a trial emulation report comprising protocol specifications, statistical summaries, and visualizations.

### TECHNICAL FIELD

The present disclosure relates generally to clinical trial design, including emulating clinical trials using multi-agent systems and real-world data.

### BACKGROUND

Clinical trials are used for evaluating the safety and efficacy of new treatments. However, traditional randomized controlled trials can be resource-intensive and may have limited generalizability due to stringent eligibility criteria.

### BRIEF DESCRIPTION OF THE SEVERAL VIEWS OF THE DRAWINGS

In the drawings, which are not necessarily drawn to scale, like numerals may describe similar components in different views. To easily identify the discussion of any particular element or act, the most significant digit or digits in a reference number refer to the figure number in which that element is first introduced. Some non-limiting examples are illustrated in the figures of the accompanying drawings in which:

FIG. 1 is a block diagram showing a clinical trial design system in accordance with some examples.

FIG. 2 is a block diagram illustrating further details regarding the clinical trial design system according to some examples.

FIG. 3 is a schematic diagram illustrating data structures, which may be stored in the database of the clinical trial design server, according to some examples.

FIG. 4 is a block diagram illustrating an architecture for data flow through the clinical trial design system according to some examples.

FIG. 5 is a diagram illustrating a multi-agent framework including tasks and agent expertise in the multi-agent orchestration system according to some examples.

FIG. 6 is a diagram illustrating an agent interaction diagram showing the operational workflow of the multi-agent orchestration system according to some examples.

FIG. 7 is a diagram illustrating structured representation of the trial knowledge graph generated by the trialist agent, according to some examples.

FIG. 8 is a diagram illustrating a criterion parsing diagram showing the process of parsing and standardizing an eligibility criterion according to some examples.

FIG. 9 is a flowchart illustrating a process for clinical trial emulation using multi-agent learning, in accordance with some examples.

FIG. 10 is a diagrammatic representation of a machine in the form of a computer system within which a set of instructions may be executed for causing the machine to perform any one or more of the methodologies discussed herein, in accordance with some examples.

FIG. 11 is a block diagram showing a software architecture within which examples may be implemented.

### DETAILED DESCRIPTION

Clinical trials provide essential data on the safety and efficacy of new treatments. When researchers design a clinical trial, they determine eligibility criteria, treatment strategies, and outcome measures. Target trial emulation has emerged as a methodology for generating real-world evidence by emulating randomized controlled trials using observational data from electronic health records. However, traditional target trial emulation processes require substantial domain expertise from clinical experts, data engineers, epidemiologists, and statisticians. These experts collaborate through extensive conversations and iterative adjustments to define trial protocols, map trial information to electronic health record elements, select appropriate covariates, apply causal inference methods, and interpret results. This manual process is time-consuming, resource-intensive, and prone to inconsistencies.

The disclosed embodiments provide a clinical trial design system that automates the target trial emulation process using a multi-agent framework. The clinical trial design system implements multiple specialized agents-including a supervisor agent, trialist agent, informatician agent, clinician agent, and statistician agent. Each agent is powered by large language models and equipped with domain-specific capabilities. The supervisor agent coordinates the workflow and synthesizes inputs from other agents. The trialist agent retrieves and standardizes clinical trial data (e.g., from registries such as ClinicalTrials.gov) and parses eligibility criteria, treatments, and outcomes into structured representations compatible with observational medical outcomes partnership common data model (OMOP-CDM) schemas. The informatician agent generates executable database queries to retrieve patient clinical data from electronic health record databases and constructs analysis-ready datasets. The clinician agent provides domain expertise by analyzing medical literature and validating trial designs. The statistician agent conducts statistical analyses using causal inference methods such as propensity score matching and inverse probability weighting to estimate treatment effects. Through iterative communication among agents and reinforcement learning with human feedback, the system refines trial protocols and generates comprehensive reports including protocol specifications, statistical summaries, and visualizations.

By automating the target trial emulation process in this manner, the clinical trial design system enhances efficiency, accuracy, and reproducibility of clinical trial designs. The clinical trial design system reduces the time and expertise required to emulate clinical trials from weeks or months to hours or days. The system facilitates exploration of multiple trial design variations by enabling rapid iteration and comparison of different eligibility criteria, treatment strategies, and outcome definitions. This automated approach reduces computational resources and processing time compared to traditional manual processes that require coordination among multiple human experts through extended consultation cycles. The system also improves transparency by documenting the complete workflow and generating standardized reports that align with clinical trial reporting standards.

### Networked Computing Environment

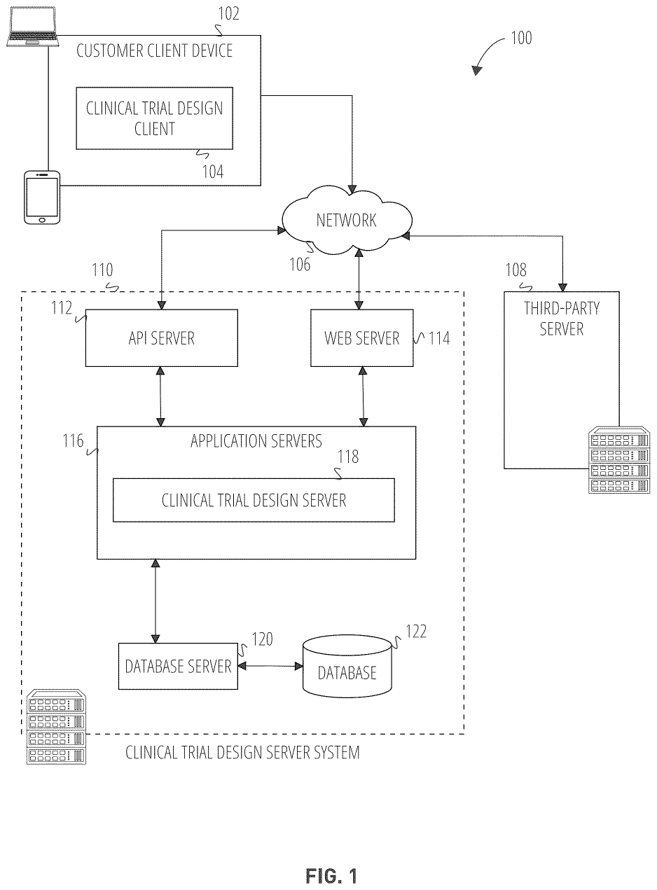

FIG. 1 is a block diagram showing a clinical trial design system 100 in accordance with some examples. The clinical trial design system 100 can include multiple instances of a customer client device 102 and multiple instances of a third-party server 108.

The customer client device 102 is associated with a client of the clinical trial design system 100. Examples of clients include pharmaceutical companies, research institutions, clinical trial organizations, healthcare analytics companies, etc. A clinical trial designer, researcher, clinician, or other employee can be the user of the customer client device 102.

Each of the customer client devices 102 hosts a number of applications, including a clinical trial design client 104. Each clinical trial design client 104 is communicatively coupled with a clinical trial design server system 110 and third-party servers 108 via a network 106 (e.g., communication network or the Internet). A clinical trial design client 104 can also communicate with locally-hosted applications using Applications Program Interfaces (APIs). The customer client devices 102 can also host a number of applications including Internet browsing applications (e.g., Chrome, Safari, etc.). The clinical trial design client 104 can also be implemented as a platform that is accessed by the customer client device 102 via an Internet browsing application or implemented as an extension on the Internet browsing application.

A clinical trial design client 104 is able to communicate and exchange data with the clinical trial design server system 110 via the network 106. The data exchanged between the clinical trial design client 104 and the clinical trial design server system 110, includes functions (e.g., commands to invoke functions) as well as payload data (e.g., target trial specifications, patient clinical data, trial emulation reports, protocol feasibility assessments, statistical summaries, visualizations, and feedback on the results and reporting provided).

The clinical trial design server system 110 can also communicate and exchange data with third-party server 108 to obtain further data and information. This includes electronic health record databases, medical literature, historical trial results, and standardized clinical ontologies (e.g., OMOP-CDM schemas, UMLS codes). The third-party server 108 can be one or more servers hosting different websites and databases including this data and information, such as ClinicalTrials.gov, PubMed, and MIMIC-IV databases.

The clinical trial design server system 110 supports various services and operations that are provided to the clinical trial design client 104. Such operations include access to the functionalities of the systems in clinical trial design server system 110. These functionalities include automated extraction of trial specifications using large language models, generation of executable database queries to retrieve patient cohorts from real-world data, execution of causal inference methods for treatment effect estimation, and iterative refinement of trial designs using reinforcement learning with human feedback. Data exchanges to and from the clinical trial design server system 110 are invoked and controlled through functions available via user interfaces (UIs) of the clinical trial design client 104.

The clinical trial design server system 110 provides server-side functionality via the network 106 to a particular clinical trial design client 104. While certain functions of the clinical trial design system 100 are described herein as being performed by either the clinical trial design client 104 or by the clinical trial design server system 110, the location of certain functionality either within the clinical trial design client 104 or the clinical trial design server system 110 may be a design choice. For example, it may be technically preferable to initially deploy certain technology and functionality within the clinical trial design server system 110 but to later migrate this technology and functionality to the clinical trial design client 104 where a customer client device 102 has sufficient processing capacity.

Turning now specifically to the clinical trial design server system 110, an Application Program Interface (API) server 112 is coupled to, and provides a programmatic interface to, application servers 116. The application servers 116 are communicatively coupled to a database server 120, which facilitates access to a database 122. The database 122 stores data from the third-party server 108 and customer client device 102 to be processed by the application servers 116. Similarly, a web server 114 is coupled to the application servers 116, and provides web-based interfaces to the application servers 116. To this end, the web server 114 processes incoming network requests over the Hypertext Transfer Protocol (HTTP) and several other related protocols.

The Application Program Interface (API) server 112 receives and transmits data between the customer client device 102 and the application servers 116. Specifically, the Application Program Interface (API) server 112 provides a set of interfaces (e.g., routines and protocols) that can be called or queried by the clinical trial design client 104 in order to invoke functionality of the application servers 116. The Application Program Interface (API) server 112 exposes to the clinical trial design client 104 various functions supported by the application servers 116. These functions include extracting trial specifications using large language models, generating structured trial definitions compatible with OMOP-CDM schemas, creating executable database queries, performing causal inference analysis using methods such as propensity score matching and survival analysis, generating protocol feasibility assessments, and providing statistical summaries and visualizations in trial emulation reports.

The application servers 116 host a number of server applications and subsystems, including for example a clinical trial design server 118. The clinical trial design server 118 implements a number of data processing technologies and functions. These include processing target trial specifications including eligibility criteria, treatments, and outcomes. The clinical trial design server 118 extracts key components and associated numerical and temporal information from these specifications. It generates structured and standardized target trial definitions compatible with OMOP-CDM schemas.

The clinical trial design server 118 then generates executable database queries based on these definitions and retrieves patient clinical data from electronic health record databases to produce analysis-ready datasets. The clinical trial design server 118 performs data quality assurance, covariate balancing, and survival analysis on these datasets using causal inference methods to estimate treatment effects. The clinical trial design server 118 obtains feedback for refining clinical parameters and covariate selections. It iteratively refines the target trial definitions, analysis-ready datasets, and treatment effect estimates by dynamically communicating this feedback and optimizing outputs via reinforcement learning with human feedback (RLHF) using a multi-agent system including a plurality of neural network models. This process generates trial emulation reports including protocol specifications, statistical summaries, and visualizations.

To perform these functions, the clinical trial design server 118 implements machine-learning solutions, neural networks, large language models, multi-agent systems, natural language processing (NLP) techniques, and reinforcement learning with human feedback (RLHF). Other processor and memory intensive processing of data may also be performed server-side by the clinical trial design server 118, in view of the hardware requirements for such processing.

### System Architecture

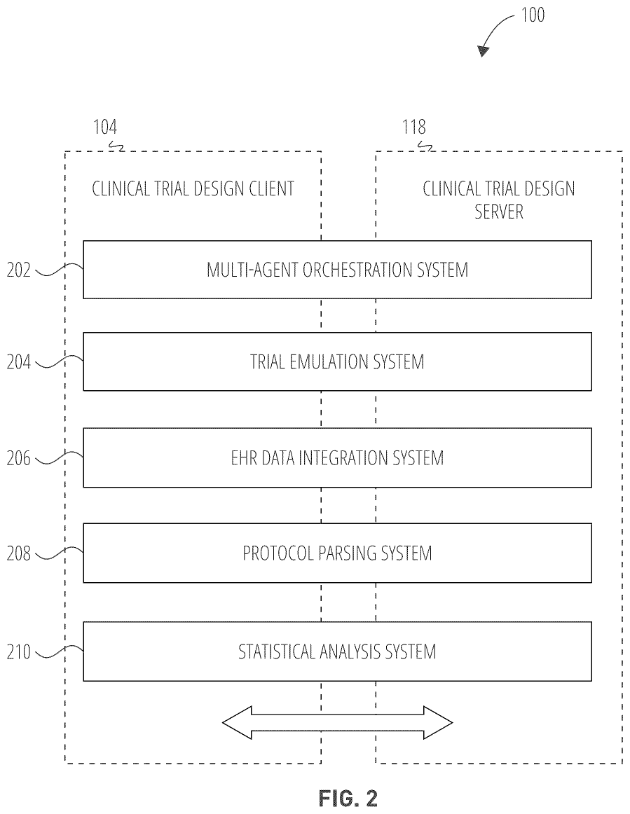

FIG. 2 is a block diagram illustrating further details regarding the clinical trial design system 100 according to some examples. Specifically, the clinical trial design system 100 is shown to include the clinical trial design client 104 and the clinical trial design server 118. The clinical trial design system 100 embodies a number of subsystems, which are supported on the client-side by the clinical trial design client 104 and on the server-side by the clinical trial design server 118. These subsystems include, for example, a multi-agent orchestration system 202, a trial emulation system 204, an Electronic Health Record (EHR) data integration system 206, a protocol parsing system 208, and a statistical analysis system 210.

The multi-agent orchestration system 202 is responsible for coordinating interactions between multiple specialized agents to perform clinical trial design and analysis tasks. The multi-agent orchestration system 202 implements a plurality of neural network models, where each model corresponds to a specialized agent with domain-specific expertise. These agents include a supervisor agent for central decision-making and task orchestration, a trialist agent for retrieving and standardizing trial information, an informatician agent for mapping trial information to EHR data structures, a statistician agent for performing statistical and causal analyses, and a clinician agent for providing domain expertise and feedback. The multi-agent orchestration system 202 coordinates communication between these agents and iteratively refines outputs using reinforcement learning with human feedback (RLHF). The multi-agent orchestration system 202 can receive input from the customer client device 102 and from the third-party server 108.

The trial emulation system 204 is responsible for generating and executing trial emulation analyses based on target trial specifications and real-world data. The trial emulation system 204 can receive target trial specifications from the customer client device 102 that describe the eligibility criteria, treatments, outcomes, and other parameters for a proposed clinical trial. The trial emulation system 204 works in conjunction with the multi-agent orchestration system 202 and other subsystems to extract key trial components, retrieve relevant patient cohorts from EHR databases, perform statistical analyses, and generate trial emulation reports. These reports include protocol specifications, statistical summaries, treatment effect estimates, and visualizations that assess the feasibility and expected outcomes of the proposed trial design.

The EHR data integration system 206 is responsible for interfacing with electronic health record databases to retrieve and standardize patient clinical data and assure data quality. The EHR data integration system 206 can receive structured target trial definitions and generate executable database queries compatible with standardized clinical data formats such as OMOP-CDM schemas. The EHR data integration system 206 retrieves patient clinical data from the third-party server 108, which hosts EHR databases such as Medical Information Mart for Intensive Care (MIMIC-IV) for acute conditions in intensive care settings and INSIGHT Clinical Research Network for chronic diseases with long-term follow-up and longitudinal patient histories. Other real-world data sources may also be accessed. The EHR data integration system 206 processes the retrieved data to produce analysis-ready datasets that are stored in the database 122 and provided to the statistical analysis system 210 for further processing.

The protocol parsing system 208 is responsible for extracting and standardizing trial protocol information from natural language descriptions. The protocol parsing system 208 utilizes large language models and natural language processing (NLP) techniques to parse target trial specifications received from the customer client device 102. The protocol parsing system 208 extracts key components including eligibility criteria, intervention treatments, outcome measures, and associated numerical and temporal information. The protocol parsing system 208 generates structured and standardized target trial definitions that map trial concepts to standardized clinical ontologies and are compatible with the schemas of common data models (CDM) such as OMOP and PCORnet. The protocol parsing system 208 can also retrieve and standardize information from historical trial protocols and medical literature obtained from the third-party server 108.

The statistical analysis system 210 is responsible for performing covariate balancing, and survival analysis to estimate treatment effects. The statistical analysis system 210 receives analysis-ready datasets from the EHR data integration system 206 and applies statistical analysis methods including propensity score matching, inverse probability of treatment weighting (IPTW), Cox proportional hazards models, random survival forest, doubly robust estimation, and others. The statistical analysis system 210 generates treatment effect estimates, statistical summaries, confidence intervals, and visualizations. The statistical analysis system 210 works in conjunction with the multi-agent orchestration system 202 to obtain feedback for refining clinical parameters and covariate selections, and iteratively improves the quality and accuracy of statistical analyses.

### Data Architecture

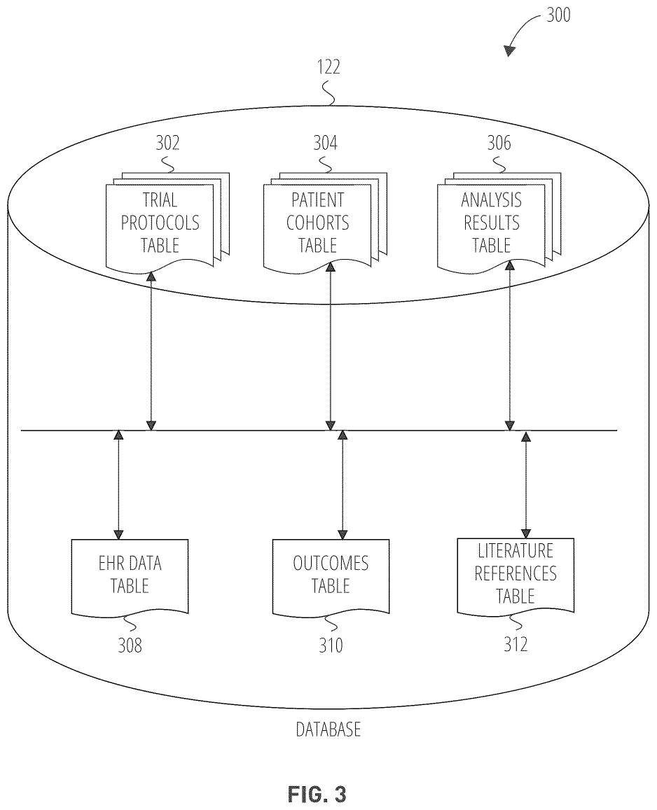

FIG. 3 is a schematic diagram illustrating data structures 300, which may be stored in the database 122 of the clinical trial design server 118, according to some examples. While the content of the database 122 is shown to include a number of tables, it will be appreciated that the data could be stored in other types of data structures (e.g., as an object-oriented database). The database 122 includes a trial protocols table 302, a patient cohorts table 304, an analysis results table 306, an Electronic Health Record (EHR) data table 308, an outcomes table 310, and a literature references table 312.

The trial protocols table 302 stores data related to target trial specifications and standardized trial definitions. For example, the trial protocols table 302 stores target trial specifications received from the customer client device 102 describing eligibility criteria, treatments, outcomes, and other trial parameters. The trial protocols table 302 also stores structured and standardized target trial definitions generated by the protocol parsing system 208 that are compatible with OMOP-CDM schemas. The trial protocols table 302 further stores extracted key components including numerical and temporal information associated with trial specifications, as well as mappings to standardized clinical ontologies obtained from third-party server 108.

The patient cohorts table 304 stores data related to patient populations retrieved from electronic health record databases. For example, the patient cohorts table 304 stores executable database queries generated by the EHR data integration system 206 and the resulting patient clinical data retrieved from third-party server 108. The patient cohorts table 304 also stores analysis-ready datasets produced after data quality assurance and preprocessing. The patient cohorts table 304 further stores cohort characteristics, eligibility status determinations, and covariate information for patients included in or excluded from trial emulation analyses.

The analysis results table 306 stores data generated by statistical and causal inference analyses performed by the statistical analysis system 210. For example, the analysis results table 306 stores treatment effect estimates, hazard ratios, confidence intervals, and p-values generated through causal inference methods such as propensity score matching, inverse probability weighting, and Cox proportional hazards models. The analysis results table 306 also stores covariate balance assessments, survival curves, and other statistical summaries. The analysis results table 306 further stores trial emulation reports including protocol specifications, statistical summaries, and visualizations generated for presentation to users via the customer client device 102.

The EHR data table 308 stores raw and processed electronic health record data obtained from external databases. For example, the EHR data table 308 stores patient clinical data retrieved from third-party server 108 including databases such as Medical Information Mart for Intensive Care (MIMIC-IV) and other real-world data sources. The EHR data table 308 also stores standardized clinical data formatted according to OMOP-CDM schemas, including condition occurrences, drug exposures, procedure occurrences, measurements, and observations. The EHR data table 308 further stores temporal information associated with clinical events and linkages between clinical concepts and standardized ontology codes.

The outcomes table 310 stores data related to clinical outcomes and endpoints for trial emulation analyses. For example, the outcomes table 310 stores outcome definitions extracted from target trial specifications including primary and secondary endpoints. The outcomes table 310 also stores observed outcome events for patients in analysis cohorts, including time-to-event data for survival analyses. The outcomes table 310 further stores outcome measurements and associated timestamps that enable temporal analysis of treatment effects.

The literature references table 312 stores data related to medical literature and historical trial results obtained from third-party server 108. For example, the literature references table 312 stores publications from databases such as PubMed and ClinicalTrials.gov that are relevant to target trial specifications. The literature references table 312 also stores historical trial protocols, published results, and metadata that inform trial design decisions. The literature references table 312 further stores citations and reference information used by the multi-agent orchestration system 202 to provide evidence-based recommendations during trial emulation processes.

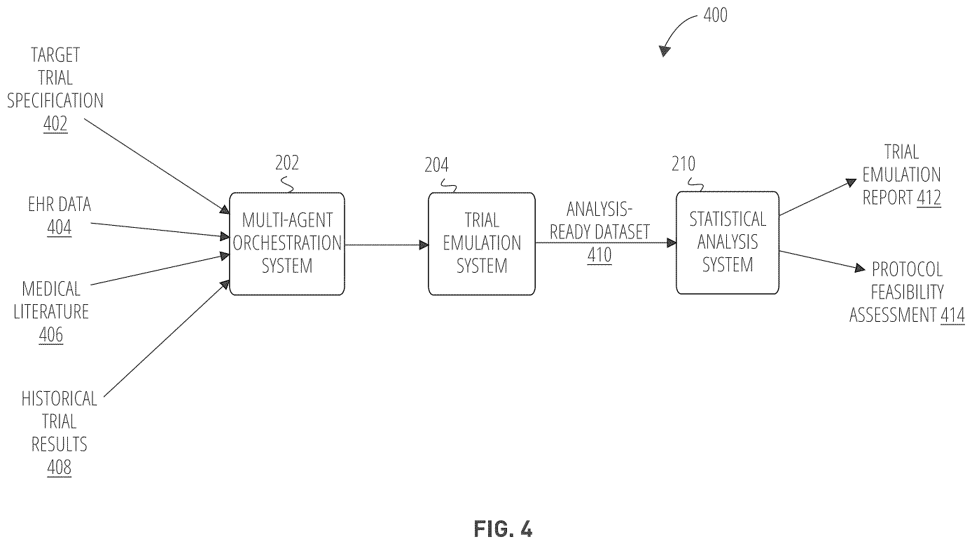

FIG. 4 is a block diagram illustrating an architecture 400 for data flow through the clinical trial design system 100 according to some examples. The architecture 400 demonstrates how various input data sources are processed by multiple subsystems to generate outputs for clinical trial design and evaluation.

As shown in the example of FIG. 4, the architecture 400 includes the multi-agent orchestration system 202, the trial emulation system 204, and the statistical analysis system 210 of FIG. 2. While not shown in FIG. 4, the trial emulation system 204 may access or otherwise interact with the EHR data integration system 206 and the protocol parsing system 208 of FIG. 2.

In example aspects, the architecture 400 is configured to receive a target trial specification 402 as input. The target trial specification 402 includes trial eligibility criteria, treatments, outcomes, and other trial parameters described in natural language. The target trial specification 402 may be received from the customer client device 102 as described with respect to FIG. 1, and stored within the trial protocols table 302 of the database 122.

In example aspects, the architecture 400 is further configured to receive EHR data 404 as input. The EHR data 404 includes patient clinical data from real-world databases such as Medical Information Mart for Intensive Care (MIMIC-IV). The EHR data 404 may be retrieved from third-party server 108 as described with respect to FIG. 1, and transmitted over network 106 to the clinical trial design server system 110 for processing. The EHR data 404 may be stored within the EHR data table 308 of the database 122.

In example aspects, the architecture 400 is further configured to receive medical literature 406 as input. The medical literature 406 includes published research articles, systematic reviews, and clinical guidelines from databases such as PubMed. The medical literature 406 may be retrieved from third-party server 108 and stored within the literature references table 312 of the database 122.

In example aspects, the architecture 400 is further configured to receive historical trial results 408 as input. The historical trial results 408 include data from previously conducted clinical trials obtained from sources such as ClinicalTrials.gov. The historical trial results 408 may be retrieved from third-party server 108 and stored within the literature references table 312 of the database 122.

As noted above, the multi-agent orchestration system 202 operates in conjunction with the trial emulation system 204 to coordinate the processing of these input data sources across multiple specialized agents. The multi-agent orchestration system 202 receives the target trial specification 402, medical literature 406, and historical trial results 408 to extract and standardize trial information, map trial concepts to standardized ontologies, and provide expert guidance throughout the trial design process. The multi-agent orchestration system 202 implements a plurality of neural network models including specialized agents with domain-specific expertise, including a supervisor agent, a trialist agent, an informatician agent, a statistician agent, and a clinician agent.

In example aspects, the supervisor agent orchestrates task delegation and manages communication between agents. The trialist agent retrieves and standardizes trial information from medical literature 406 and historical trial results 408. The informatician agent maps trial concepts to standardized clinical terminologies and Electronic Health Record (EHR) data structures compatible with OMOP-CDM schemas. The statistician agent specifies appropriate statistical methods and validates analytical approaches. The clinician agent provides domain expertise to ensure clinical relevance and identifies potential safety concerns.

In example aspects, the multi-agent orchestration system 202 implements reinforcement learning with human feedback (RLHF) to iteratively refine outputs based on domain expertise and user feedback. This enables continuous improvement of trial design recommendations through iterative agent interactions.

In example aspects, the trial emulation system 204 receives data from the multi-agent orchestration system 202 and processes the target trial specification 402, EHR data 404, medical literature 406, and historical trial results 408 to generate and execute trial emulation analyses. The trial emulation system 204 works in conjunction with the protocol parsing system 208 and EHR data integration system 206 (shown in FIG. 2) to extract key trial components, generate structured target trial definitions compatible with OMOP-CDM schemas, and create executable database queries to retrieve relevant patient cohorts from EHR databases.

In example aspects, the trial emulation system 204 applies natural language processing (NLP) techniques to parse eligibility criteria, identify inclusion and exclusion conditions, extract temporal constraints, and map clinical concepts to standardized ontology codes. The trial emulation system 204 then generates Structured Query Language (SQL) queries that are executed against EHR data 404 to identify eligible patients, retrieve their clinical histories, and extract relevant covariates for adjustment in statistical analyses. The trial emulation system 204 further applies NLP techniques to analyze unstructured clinical notes from EHR data 404 to extract clinically relevant information not available in structured data fields. The NLP analysis of clinical notes identifies additional patients who meet eligibility criteria based on information documented in narrative text, extracts relevant clinical events and temporal relationships, and identifies adverse events or outcomes described in clinical documentation. The NLP pipeline processes clinical notes to augment the structured data retrieved from EHR databases, enhancing the completeness and accuracy of the analysis-ready dataset 410.

In example aspects, the trial emulation system 204 performs data quality checks to identify missing data, outliers, and inconsistencies, and applies preprocessing steps such as data normalization, feature engineering, and cohort matching. The trial emulation system 204 produces an analysis-ready dataset 410 that is stored in database 122 (shown in FIG. 3) and includes preprocessed patient clinical data with appropriate covariate information for statistical analysis. The analysis-ready dataset 410 may be stored within the patient cohorts table 304 of the database 122.

In example aspects, the trial emulation system 204 generates structured outputs for clinical trial design evaluation. These outputs include a trial emulation report and a protocol feasibility assessment. The trial emulation report provides comprehensive documentation of the emulated trial including protocol specifications, treatment effect estimates, statistical summaries, confidence intervals, and visualizations that assess the expected outcomes of the proposed trial design. The protocol feasibility assessment evaluates the viability of conducting the proposed trial based on available patient populations, anticipated enrollment rates, statistical power considerations, and operational constraints.

In example aspects, the statistical analysis system 210 in conjunction with the multi-agent orchestration system 202 and/or the trial emulation system 204 is configured to generate the trial emulation report 412 and the protocol feasibility assessment 414 based on statistical analysis of the analysis-ready dataset 410. As shown in the example of FIG. 4, the statistical analysis system 210 receives the analysis-ready dataset 410 from the trial emulation system 204.

In example aspects, the statistical analysis system 210 performs data quality assurance, covariate balancing, and survival analysis using causal inference methods. The statistical analysis system 210 implements multiple causal inference techniques to estimate treatment effects while controlling for confounding variables. The statistical analysis system 210 applies propensity score matching to create balanced treatment and control groups by matching patients with similar baseline characteristics. The statistical analysis system 210 implements inverse probability weighting to adjust for selection bias and differences in treatment assignment probabilities. The statistical analysis system 210 conducts survival analysis using Cox proportional hazards models to estimate time-to-event outcomes and generate hazard ratios comparing treatment effects.

In example aspects, the statistical analysis system 210 further implements adverse event modeling capabilities to identify and analyze safety signals in the analysis-ready dataset 410. The statistical analysis system 210 applies a two-step process for adverse event analysis. First, the statistical analysis system 210 identifies potential adverse events from clinical trial knowledge graphs and biomedical literature using the multi-agent orchestration system 202 in conjunction with the clinician agent. Second, the statistical analysis system 210 performs statistical analysis to evaluate the relationship between treatment exposure and identified adverse events, generating adverse event profiles that include event frequencies, severity classifications, and temporal patterns. The adverse event analysis results are incorporated into the trial emulation report 412 to provide comprehensive safety assessments alongside efficacy estimates.

In example aspects, the statistical analysis system 210 implements adaptive sample size calculation functionality to optimize trial resource allocation while preserving statistical power. The statistical analysis system 210 applies the Schoenfeld formula for Cox proportional hazards models to estimate required sample sizes based on empirical characteristics observed in the analysis-ready dataset 410. The sample size calculation incorporates treatment allocation ratios, baseline event rates, censoring proportions, average follow-up times, Type I error rates, desired statistical power, and expected hazard ratios derived from prior trial literature or preliminary analyses. The statistical analysis system 210 dynamically adjusts sample size recommendations based on real-world data characteristics, enabling data-driven trial planning that balances feasibility constraints with inferential requirements. The adaptive sample size estimates are included in the protocol feasibility assessment 414 to inform trial design decisions.

In example aspects, the statistical analysis system 210 implements methodologies to address immortal time bias in trial emulation analyses. Immortal time bias occurs when patients survive long enough to meet certain eligibility criteria or receive treatment, creating a period during which death is impossible by design. The statistical analysis system 210 applies clone-censor-weight methodology to properly account for immortal time. This methodology involves creating cloned patient records to represent all possible treatment trajectories, censoring clones at the time of actual treatment assignment, and applying appropriate inverse probability weights to account for treatment selection. The immortal time bias correction is automatically applied by the statistician agent when temporal eligibility criteria or treatment timing considerations are identified in the trial protocol. Results from analyses with and without immortal time bias correction are documented in the trial emulation report 412 to demonstrate the impact of this methodological consideration.

In example aspects, the statistical analysis system 210 calculates confidence intervals, performs sensitivity analyses to assess robustness of findings, and generates visualizations including Kaplan-Meier survival curves, covariate balance plots, and forest plots of treatment effects.

In example aspects, the statistical analysis system 210 is configured to store the trial emulation report 412 and the protocol feasibility assessment 414 (as defined above) in association with the respective clinical trial design. For example, the trial emulation report 412 and protocol feasibility assessment 414 are stored within the analysis results table 306 of the database 122.

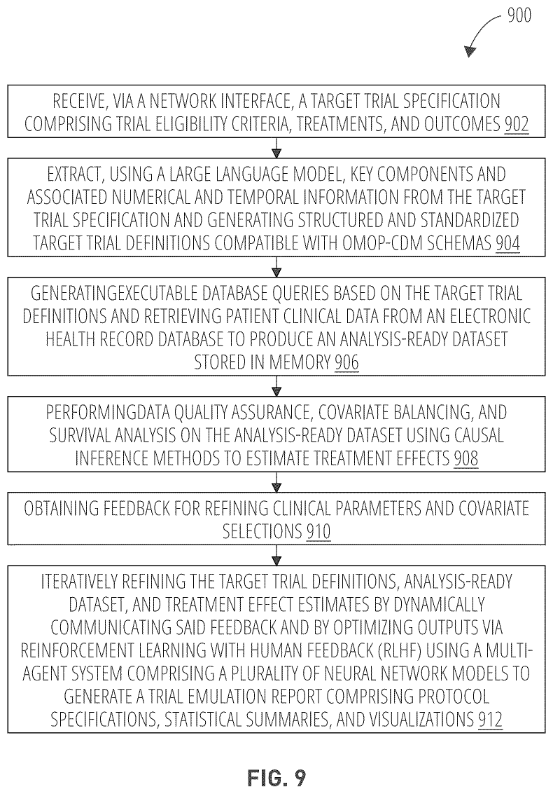

As discussed further below with respect to FIG. 9, the statistical analysis system 210 generates the trial emulation report 412 and protocol feasibility assessment 414 noted above. Thus, the architecture 400 is depicted as outputting the trial emulation report 412 and the protocol feasibility assessment 414, which are provided to the customer client device 102 for review. Thus, the architecture 400 is depicted as outputting the trial emulation report 412 and the protocol feasibility assessment 414, which are provided to the customer client device 102 for review.

In example aspects, the analysis-ready dataset 410 serves as the foundation for conducting comprehensive statistical analyses and can be used to perform additional exploratory analyses, sensitivity analyses with different statistical approaches, and subgroup analyses to identify heterogeneous treatment effects across patient populations. The trial emulation report 412 provides evidence-based insights that inform decision-making regarding whether to proceed with a prospective clinical trial, helps optimize trial design parameters such as sample size and inclusion criteria, and identifies potential challenges or limitations that should be addressed before initiating a costly prospective trial.

In example aspects, the protocol feasibility assessment 414 enables stakeholders to make informed go/no-go decisions regarding trial initiation, helps prioritize trial designs with the highest likelihood of success, and identifies modifications to eligibility criteria or recruitment strategies that may improve trial feasibility. The trial emulation report 412 and protocol feasibility assessment 414 are stored in database 122 (for example, in analysis results table 306 as shown in FIG. 3) and may be transmitted over network 106 to the customer client device 102 for review and further action by clinical trial designers, researchers, and other stakeholders involved in the trial planning process.

Thus, the architecture 400 employs neural network model(s) and multi-agent systems in analyzing target trial specifications, EHR data, medical literature, and historical trial results, in order to generate trial emulation reports and protocol feasibility assessments for clinical trial designers. The architecture 400 provides user interfaces which allow an end user (e.g., a clinical trial designer or researcher) to view the trial emulation reports and associated feasibility assessments, and to manually make any modifications. Moreover, the architecture 400 provides for receiving and analyzing new target trial specifications based on feedback and iterative refinement, and for generating updated trial emulation reports for review by stakeholders.

By generating trial emulation reports and protocol feasibility assessments in this manner, the architecture 400 enhances capacity, accuracy and transparency of the clinical trial design process. Moreover, the architecture 400 facilitates the analysis of real-world data in view of proposed trial designs (e.g., typically performed manually and repetitively by clinical researchers in association with statistical analysis software), thereby saving time for end users, and reducing computational resources/processing power.

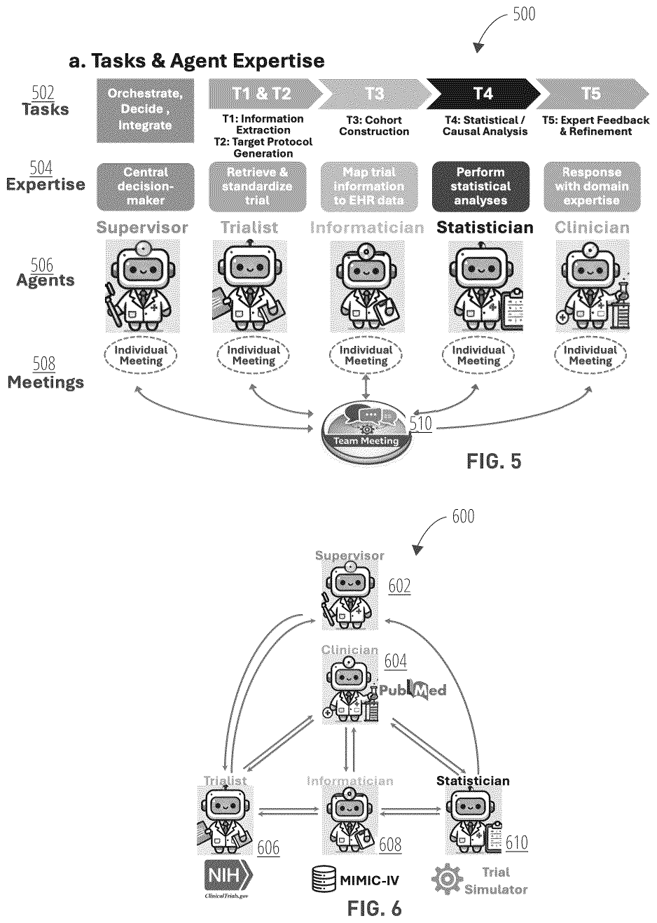

FIG. 5 is a diagram illustrating a multi-agent framework 500 including tasks and agent expertise in the multi-agent orchestration system 202 according to some examples. The multi-agent framework 500 demonstrates the division of responsibilities among specialized agents and the types of collaborative meetings that facilitate clinical trial design and emulation workflows.

As shown in the example of FIG. 5, the multi-agent framework 500 illustrates tasks 502, expertise 504, agents 506, and meetings 508 and 510. The tasks 502 section defines five core tasks performed within the clinical trial design system 100. The expertise 504 section describes the domain-specific capabilities assigned to each agent. The agents 506 section identifies the five specialized agents that include the multi-agent orchestration system 202. The meetings 508 and 510 sections illustrate the collaborative frameworks through which agents interact.

In example aspects, the tasks 502 include a comprehensive workflow for clinical trial design. Task T1 involves information extraction, wherein relevant clinical trial data is retrieved from external sources and parsed into structured formats. Task T2 involves target protocol generation, wherein standardized trial protocols are created based on extracted information. Task T3 involves cohort construction, wherein patient populations are identified and datasets are built from EHR data. Task T4 involves statistical and causal analysis, wherein treatment effects are estimated using causal inference methods. Task T5 involves expert feedback and refinement, wherein clinical domain expertise is applied to validate and improve trial designs. An additional orchestration function involves coordinating tasks, making critical decisions, and integrating outputs from all agents.

In example aspects, the expertise 504 defines domain-specific capabilities for each specialized agent. The supervisor agent possesses expertise as a central decision-maker that orchestrates workflow and synthesizes inputs from all agents. The trialist agent possesses expertise in retrieving and standardizing trial information from registries such as ClinicalTrials.gov and medical literature databases. The informatician agent possesses expertise in mapping trial information to EHR data structures and generating executable database queries compatible with OMOP-CDM schemas. The statistician agent possesses expertise in performing statistical analyses using causal inference methods to estimate treatment effects. The clinician agent possesses expertise in providing domain knowledge regarding clinical validity, covariate selection, and interpretation of results.

In example aspects, the agents 506 include five specialized neural network models that implement the multi-agent orchestration system 202 as described with respect to FIG. 2. The supervisor agent serves as the central coordinator, managing workflow and ensuring seamless communication between other agents. The supervisor agent dynamically assigns tasks and determines when to initiate or terminate workflows. The supervisor agent synthesizes inputs from all agents and generates comprehensive reports. The supervisor agent also logs all operations, decisions, and inter-agent communications to ensure transparency and reproducibility.

In example aspects, the trialist agent autonomously retrieves and standardizes clinical trial data from registries including ClinicalTrials.gov and PubMed. The trialist agent extracts key components of target trials including eligibility criteria, treatment assignments, and outcome measures. The trialist agent applies natural language processing (NLP) techniques to parse trial protocols and associated numerical and temporal information. The trialist agent generates structured and standardized trial definitions compatible with OMOP-CDM schemas. The trialist agent annotates trial information using the Unified Medical Language System (UMLS) for standardization. The trialist agent performs tasks T1 and T2 by extracting clinical trial information and generating target trial protocols.

In example aspects, the informatician agent bridges trial information with real-world data by matching trial eligibility criteria to EHR data and constructing datasets for analysis. The informatician agent translates inclusion and exclusion criteria, treatment assignments, and outcome measures into executable SQL queries. The informatician agent may use tools including large language models to generate the SQL queries for data extraction. The informatician agent performs data quality assurance by evaluating completeness, logical consistency, and clinical plausibility of constructed datasets. The informatician agent performs rigorous checks to identify missing data, outliers, and inconsistencies. The informatician agent applies preprocessing steps including data normalization and feature engineering. The informatician agent produces analysis-ready datasets containing patient-level rows with identifiers, eligibility flags, treatment indicators, outcome metrics, and baseline covariates. The informatician agent performs task T3 by constructing cohorts and building datasets from EHR data.

In example aspects, the statistician agent performs target trial emulation by selecting causal inference methods, conducting survival analyses, and interpreting results. The statistician agent implements multiple causal inference techniques including Propensity Score Matching (PSM), Inverse Probability of Treatment Weighting (IPTW), Cox regression, parametric survival models, and random survival forests (RSF). The statistician agent conducts survival analysis using Cox proportional hazards models to evaluate time-to-event outcomes and generate hazard ratios comparing treatment effects. The statistician agent calculates confidence intervals and performs sensitivity analyses to assess robustness of findings. The statistician agent generates visualizations including Kaplan-Meier survival curves, covariate balance plots, and forest plots of treatment effects. The statistician agent synthesizes findings into cohesive reports including result summaries, adjusted hazard ratios, confidence intervals, and p-values. The statistician agent performs task T4 by conducting statistical and causal analyses.

In example aspects, the clinician agent provides domain expertise for validating trial design and addressing discrepancies in trial emulation through iterative interactions with other agents. The clinician agent retrieves and synthesizes medical literature using a retrieval-augmented generation (RAG) approach to provide evidence-based recommendations. The clinician agent performs semantic searches over biomedical knowledge bases including PubMed to identify relevant clinical guidelines and research findings. The clinician agent identifies relevant covariates for conducting trial emulation and validates trial designs against medical evidence. The clinician agent provides recommendations for substituting missing covariates with clinically equivalent variables. The clinician agent delivers responses in standardized, machine-readable formats using annotations such as tags for conditions, drugs, measurements, and temporal modifiers. The clinician agent performs task T5 by providing expert feedback and refinement throughout the trial design process.

In example aspects, the agents 506 are trained via reinforcement learning with human feedback (RLHF) to iteratively improve the quality of outputs. The trialist agent is trained via RLHF to improve parsing fidelity and compatibility with OMOP-CDM schemas. The informatician agent is trained via RLHF to produce executable database queries. The statistician agent is trained via RLHF on statistical validity, interpretability, and alignment with study objectives. The clinician agent is trained via RLHF to generate clinically grounded suggestions regarding covariate selection, surrogate variable mapping, and eligibility relaxation strategies.

In example aspects, the meetings 508 and 510 represent collaborative frameworks through which agents share expertise, refine tasks, and ensure high-quality outputs. Meetings are categorized into individual meetings 508 and team meetings 510. The meetings include defined components such as agenda, agenda questions, agenda rules, summaries, contexts, and rounds. The agenda provides a clear description of the topic to be discussed or task to be accomplished. Agenda questions specify questions to be addressed by the agents during the meeting. Agenda rules define constraints or guidelines that agents follow when discussing or executing tasks. Summaries provide optional summaries of prior meetings or decisions to provide context. Contexts provide supplementary information such as trial protocols or dataset summaries that agents may reference. Rounds define the number of discussion or iteration rounds, typically set by the supervisor agent.

In example aspects, the individual meetings 508 focus on task-specific activities typically assigned to a single agent, with optional feedback from other agents or the supervisor agent. Individual meetings allow for in-depth execution and refinement of specialized tasks such as coding SQL queries or running survival analysis models. For example, an individual meeting may involve the informatician agent tasked with generating a dataset based on updated eligibility criteria. The agenda specifies the need to construct SQL queries that incorporate relaxed temporal conditions for diagnosis criteria. The informatician agent writes and reviews the queries. The informatician agent identifies potential issues such as unanticipated data gaps and receives targeted feedback from the supervisor agent to address these gaps. After iterative revisions, the informatician agent submits the final dataset accompanied by a summary of modifications and justifications. Individual meetings ensure that agents can dedicate attention to complex tasks while receiving support and oversight as needed.

In example aspects, the team meetings 510 bring together all agents to address complex, high-level issues requiring interdisciplinary expertise. Team meetings are organized by the supervisor agent, who sets the meeting agenda and synthesizes inputs from all agents. The discussions typically revolve around broad questions such as optimizing trial eligibility criteria or selecting analytical methods. For example, a team meeting may be focused on addressing high missing rates in laboratory values within a dataset. The supervisor agent initiates the discussion by outlining the agenda to assess whether surrogate variables can replace the missing values. The informatician agent presents data on the extent of missingness and feasibility of implementing surrogate measures. The clinician agent evaluates the clinical validity of proposed substitutes by referencing medical literature from PubMed or other sources. The statistician agent weighs the statistical implications, particularly the impact on covariate balancing. The discussion unfolds over multiple rounds with agents refining their responses based on feedback. The supervisor agent consolidates the insights, approves the proposed approach, and assigns follow-up tasks to agents for implementation. Team meetings are essential for resolving ambiguities and achieving consensus on critical decisions.

In example aspects, the multi-agent orchestration system 202 implements a modular framework wherein each agent 506 is assigned distinct responsibilities corresponding to specific tasks 502 and leverages specialized expertise 504. This modular design reflects the natural division of labor in clinical research teams and enables agents to iterate collaboratively. Agents dynamically refine eligibility definitions, propose surrogate covariates, and adjust statistical models. This mirrors the interdisciplinary nature of clinical trial design in practice. The multi-agent orchestration system 202 supports both a core sequential pipeline (supervisor agent to trialist agent to informatician agent to clinician agent to statistician agent to supervisor agent) and dynamic interactions between agents. For instance, the informatician agent can query the clinician agent when facing data sparsity or missing covariates. This prompts iterative refinements in eligibility criteria or variable substitutions.

In example aspects, the meetings 508 and 510 enable the multi-agent orchestration system 202 to address challenges that arise during trial design and emulation. Through individual meetings 508, agents execute specialized tasks with focused attention and receive targeted feedback. Through team meetings 510, agents leverage diverse expertise to navigate complexities and make informed decisions collaboratively. These collaborative frameworks ensure that the multi-agent orchestration system 202 continuously validates and optimizes trial designs, providing robust and actionable insights for clinical trial designers and researchers.

Thus, the multi-agent framework 500 illustrates how tasks, expertise, agents, and meetings are organized within the multi-agent orchestration system 202 to facilitate efficient, collaborative, and iterative clinical trial design workflows. The division of tasks 502 among specialized agents 506 with distinct expertise 504, combined with structured meetings 508 and 510, enables the clinical trial design system 100 to transform the traditionally manual, expert-driven process of clinical trial design into a scalable, intelligent, and collaborative workflow.

FIG. 6 is a diagram illustrating an agent interaction diagram 600 showing the operational workflow of the multi-agent orchestration system 202 according to some examples. The agent interaction diagram 600 demonstrates an example of how the supervisor agent 602 coordinates interactions among specialized agents and how each agent connects to external tools and data sources to execute clinical trial design and emulation tasks.

As shown in the example of FIG. 6, the agent interaction diagram 600 illustrates the supervisor agent 602 positioned at the center with connections to the clinician agent 604, the trialist agent 606, the informatician agent 608, and the statistician agent 610. The agents 602, 604, 606, 608, and 610 correspond to the agents 506 described with respect to FIG. 5. Each specialized agent is further connected to external tools and data sources that provide domain-specific capabilities. The clinician agent 604 connects to PubMed for retrieving medical literature. The trialist agent 606 connects to NIH ClinicalTrials.gov for retrieving trial protocols. The informatician agent 608 connects to MIMIC-IV for accessing EHR data. The statistician agent 610 connects to Trial Simulator for conducting statistical analyses.

In example aspects, the supervisor agent 602 serves as the central coordinator managing workflow and orchestrating interactions among all specialized agents. The supervisor agent 602 receives input from the customer client device 102 including the target trial specification 402 as described with respect to FIG. 4. The supervisor agent 602 synthesizes inputs from the human user and determines the start or end of the workflow. The supervisor agent 602 dynamically assigns tasks to specialized agents based on the current state of the clinical trial design process. The supervisor agent 602 coordinates communication between agents by facilitating individual meetings 508 and team meetings 510 as described with respect to FIG. 5. The supervisor agent 602 logs all operations, decisions, and inter-agent communications to ensure transparency and reproducibility. The supervisor agent 602 consolidates insights from all agents and generates the final trial emulation report 412.

In example aspects, the trialist agent 606 autonomously retrieves and standardizes clinical trial data (e.g., from NIH ClinicalTrials.gov). The connection between the trialist agent 606 and NIH ClinicalTrials.gov enables construction of a clinical trial knowledge graph containing complete information of clinical trials. The trialist agent 606 applies NLP techniques to parse trial protocols and associated numerical and temporal information for trials from ClinicalTrials.gov. The trialist agent 606 annotates trial information using the Unified Medical Language System (UMLS) for standardization. The trialist agent 606 generates structured and standardized trial definitions compatible with OMOP-CDM schemas. The trialist agent 606 maps clinical concepts to standardized vocabularies including condition (e.g., ICD-9/ICD-10), drug (e.g., RxNorm), measurement (e.g., LOINC), and procedure (e.g., SNOMED CT). The trialist agent 606 constructs a clinical trial knowledge graph containing the structured and standardized information of trials from NIH ClinicalTrials.gov. The trialist agent 606 identifies relevant trials for a given study question by performing queries with complex conditions over the knowledge graph. The trialist agent 606 extracts key components of target trials including eligibility criteria, treatment assignments, and outcome measures. The trialist agent 606 provides the necessary information for the informatician agent 608 to generate SQL queries for retrieving relevant information from EHR data.

In example aspects, the informatician agent 608 bridges trial information with real-world data by connecting to MIMIC-IV and INSIGHT Clinical Research Network. The connection between the informatician agent 608 and MIMIC-IV enables access to high-resolution, time-stamped clinical data essential for modeling rapid disease progression in acute care settings. The connection to INSIGHT Clinical Research Network enables access to longitudinal patient histories spanning extended time horizons, supporting the evaluation of chronic disease trajectories including conditions such as Alzheimer's disease and Parkinson's disease. The informatician agent 608 selects the appropriate data source based on the clinical characteristics of the target trial, utilizing MIMIC-IV for acute conditions requiring intensive care data and INSIGHT for chronic diseases requiring long-term follow-up data.

The informatician agent 608 translates inclusion and exclusion criteria, treatment assignments, and outcome measures into executable SQL queries. The informatician agent 608 may use tools including large language models to generate the SQL queries for data extraction. The informatician agent 608 renders each criterion as a Common Table Expression (CTE), allowing for modular and sequential cohort construction. The informatician agent 608 executes SQL queries to retrieve relevant patient data and merges extracted tables to form a comprehensive patient dataset. The informatician agent 608 performs data quality assurance by evaluating completeness, logical consistency, and clinical plausibility of constructed datasets. The informatician agent 608 performs rigorous checks to identify missing data, outliers, and inconsistencies. The informatician agent 608 applies preprocessing steps including data normalization and feature engineering. The informatician agent 608 produces analysis-ready datasets containing patient-level rows with identifiers, eligibility flags, treatment indicators, outcome metrics, and baseline covariates. The informatician agent 608 stores the analysis-ready dataset 410 in database 122 for access by the statistician agent 610.

In example aspects, the informatician agent 608 incorporates NLP techniques to analyze unstructured clinical notes. The informatician agent 608 processes clinical notes to identify additional eligible patients who might be missed by queries limited to structured fields alone. Clinical notes contain a wealth of information regarding patient history, conditions, and observations that are often not codified. The informatician agent 608 can identify additional eligible patients through NLP of clinical notes that are not discoverable via structured EHR data alone.

In example aspects, the statistician agent 610 performs target trial emulation by connecting to Trial Simulator. The connection between the statistician agent 610 and Trial Simulator provides access to statistical and machine learning libraries for confounder adjustment, outcome analysis, and treatment effect estimation. The statistician agent 610 selects the best covariate balancing strategy and outcome analysis method. The statistician agent 610 evaluates multiple options for balancing including Propensity Score Matching (PSM), Inverse Probability of Treatment Weighting (IPTW), or no balancing. The statistician agent 610 bases this selection on factors such as sample size, the distribution of covariates, and the research objective of estimating causal effects. The statistician agent 610 applies the selected balancing method to create balanced treatment and control groups. The statistician agent 610 conducts survival analysis using Cox proportional hazards models to evaluate time-to-event outcomes and generate hazard ratios comparing treatment effects. The statistician agent 610 can select from Cox Proportional Hazards, Kaplan-Meier estimation, parametric survival models, random survival forests (RSF), or doubly robust methods. The statistician agent 610 calculates confidence intervals and performs sensitivity analyses to assess robustness of findings. The statistician agent 610 generates visualizations including Kaplan-Meier survival curves, covariate balance plots, and forest plots of treatment effects. The statistician agent 610 synthesizes findings into cohesive reports including result summaries, adjusted hazard ratios, confidence intervals, and p-values.

In example aspects, the statistician agent 610 can perform subgroup analyses to explore treatment heterogeneity. Following an initial survival analysis, the statistician agent 610 may detect that the estimated treatment effect is not statistically significant in the overall sample. The statistician agent 610 can prompt the clinician agent 604 to propose a covariate and threshold for splitting the cohort into two clinically relevant subgroups. The statistician agent 610 then reruns the survival model for each subgroup, comparing the hazard ratios and confidence intervals separately. This helps identify any sub-populations where the treatment might be more or less effective. The statistician agent 610 can also perform adverse event reporting and adaptive sample size calculation.

In example aspects, the clinician agent 604 provides domain expertise by connecting to PubMed for retrieving medical literature. The connection between the clinician agent 604 and PubMed enables a retrieval-augmented generation (RAG) approach to gather relevant medical literature. The clinician agent 604 performs semantic searches over a biomedical knowledge base to identify relevant clinical guidelines and research findings. The clinician agent 604 retrieves and synthesizes medical literature to provide evidence-based recommendations. The clinician agent 604 reviews reports generated by the statistician agent 610 and either recommends modifications or approves the analysis. The clinician agent 604 provides evidence-based recommendations to other agents at various stages of the trial emulation process. The clinician agent 604 identifies relevant covariates for conducting trial emulation and validates trial designs against medical evidence. The clinician agent 604 provides recommendations for substituting missing covariates with clinically equivalent variables. The clinician agent 604 delivers responses in standardized, machine-readable formats using annotations such as tags for conditions, drugs, measurements, and temporal modifiers. This structured communication allows other agents, particularly the informatician agent 608, to directly map the recommendations onto EHR queries without requiring manual interpretation.

In example aspects, the agent interaction diagram 600 implements tool-augmented reasoning capabilities for each agent. The tools are invoked automatically within the agent workflow to convert natural language insights into executable code, structured queries, and interpretable analytics. The trialist agent 606 parses trials protocols from clinical trial registries such as ClinicalTrials.gov. The clinician agent 604 uses PubMed through a RAG module to ground its decisions in biomedical literature by performing semantic searches over large corpora. The informatician agent 608 uses MIMIC-IV to access real-world EHR data for cohort construction and dataset generation. The statistician agent 610 uses Trial Simulator to access statistical and machine learning libraries for confounder adjustment, survival analysis, and treatment effect estimation.

In example aspects, the supervisor agent 602 facilitates dynamic interactions between agents beyond the core sequential pipeline. The multi-agent orchestration system 202 supports both a core sequential pipeline (supervisor agent 602 to trialist agent 606 to informatician agent 608 to clinician agent 604 to statistician agent 610 to supervisor agent 602) and dynamic interactions between agents. For instance, the informatician agent 608 can query the clinician agent 604 when facing data sparsity or missing covariates. This prompts iterative refinements in eligibility criteria or variable substitutions. If the informatician agent 608 identifies high levels of missingness for a key variable or detects poor covariate balance, the system can autonomously initiate a feedback loop with the clinician agent 604 to assess alternative variable definitions or biomedically appropriate surrogates. The statistician agent 610 can consult the clinician agent 604 when persistent imbalances are detected in covariate balancing, triggering feedback to upstream agents for further refinement or relaxation of eligibility criteria.

In example aspects, the agent interaction diagram 600 ensures that patient privacy is preserved when using external tools and data sources. The informatician agent 608 sends the database schema and trial protocol to the large language model to generate SQL queries without transmitting patient data. This architectural design ensures that no personally identifiable information (PII) or protected health information (PHI) is transmitted to external language model services. These queries are then executed locally behind the institution's firewall. This ensures that even when using proprietary cloud-based models, patient privacy is preserved. All patient-level data processing and analysis operations are performed within secure computing environments that comply with applicable privacy regulations including HIPAA (Health Insurance Portability and Accountability Act) and GDPR (General Data Protection Regulation) where applicable. The multi-agent orchestration system 202 implements data de-identification procedures to remove or encrypt patient identifiers before any data visualization or reporting functions that may be accessed by users through the clinical trial design client 104.

In example aspects, agents are trained via reinforcement learning with human feedback (RLHF) to iteratively improve the quality of outputs. The trialist agent 606 is trained via RLHF to improve parsing fidelity and compatibility with OMOP-CDM schemas. The informatician agent 608 is trained via RLHF to produce executable database queries. The statistician agent 610 is trained via RLHF on statistical validity, interpretability, and alignment with study objectives. The clinician agent 604 is trained via RLHF to generate clinically grounded suggestions regarding covariate selection, surrogate variable mapping, and eligibility relaxation strategies.

Thus, the agent interaction diagram 600 illustrates how the supervisor agent 602 coordinates specialized agents and their connections to external tools and data sources to execute clinical trial design and emulation workflows. The modular design enables each agent to leverage domain-specific tools while maintaining seamless communication through the supervisor agent 602. This architecture transforms the traditionally manual, expert-driven process of clinical trial design into an efficient, intelligent, and collaborative workflow that integrates real-world data with clinical expertise.

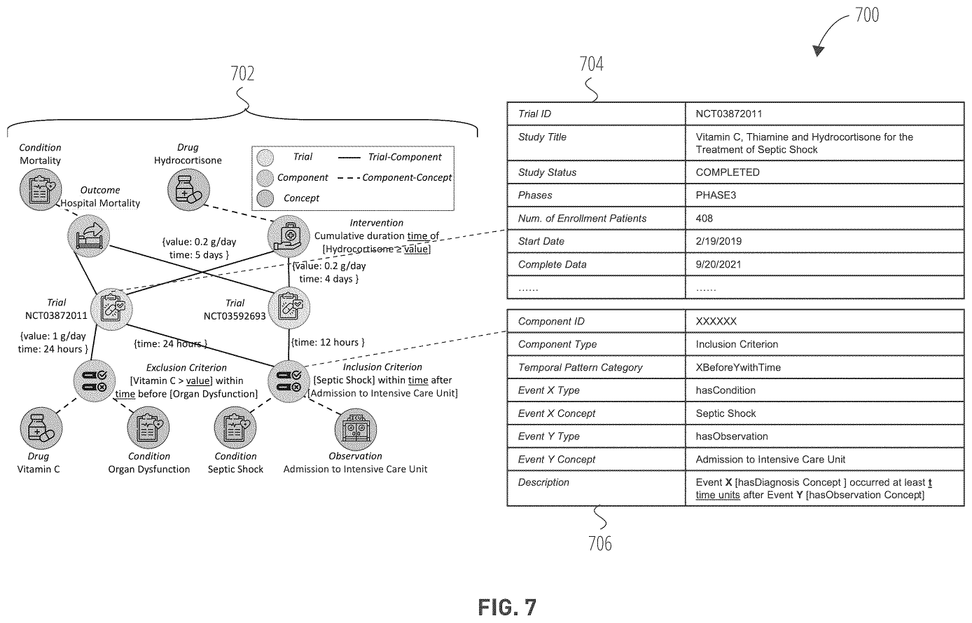

FIG. 7 is a diagram illustrating structured trial representation 700 for the trial knowledge graph generated by the trialist agent 606, according to some examples. The structured trial representation 700 demonstrates the output format produced by the trialist agent 606 after retrieving and standardizing clinical trial data from registries such as ClinicalTrials.gov. The structured trial representation 700 provides a comprehensive representation of a target clinical trial that is compatible with OMOP-CDM schemas and serves as the foundation for downstream processing by the informatician agent 608. As shown in the example of FIG. 7, the structured trial representation 700 includes graph node representation 702, trial metadata 704, and structured eligibility criteria 706.

The graph node representation 702 shows how the extracted trial information and relevant clinical concepts from the eligibility criteria are represented as nodes in the trial knowledge graph. The graph node representation 702 illustrates the relationships between the trial nodes, component nodes, and concept nodes within the knowledge graph architecture. The graph node representation 702 shows trial nodes identified by their NCT numbers such as “NCT03872011” and “NCT03592693” connected to trial component nodes through “Trial-Component” relationships. The trial component nodes represent individual eligibility criteria and interventions such as “Inclusion Criterion [Septic Shock] within time after [Admission to Intensive Care Unit],” “Exclusion Criterion [Vitamin C>value] within time before [Organ Dysfunction],” and “Intervention Cumulative duration time of [Hydrocortisone≥value].” The component nodes are linked to specific clinical concept nodes through “Component-Concept” relationships. The concept nodes represent standardized clinical concepts from OMOP-CDM vocabularies including “Condition: Septic Shock”, “Condition: Organ Dysfunction”, “Condition: Mortality”, “Observation: Admission to Intensive Care Unit”, “Drug: Vitamin C” and “Drug: Hydrocortisone”. The standardized concept mapping 702 further includes temporal and value attributes associated with the relationships such as “{time: 24 hours}”, “{time: 12 hours}”, “{value: 0.2 g/day time: 5 days}”, “{value: 0.2 g/day time: 4 days}”, and “{value: 1 g/day time: 24 hours}” indicating the temporal constraints and dosage specifications in the eligibility criteria and intervention protocols.

The trial metadata 704 provides essential identifying information and administrative details for the clinical trial. The trial metadata 704 includes the Trial ID field containing the unique clinical trial registry identifier from ClinicalTrials.gov such as “NCT03872011.” The trial metadata 704 further includes the Study Title field providing the full title of the clinical trial such as “Vitamin C, Thiamine and Hydrocortisone for the Treatment of Septic Shock.” The trial metadata 704 includes the Study Status field indicating the current status of the trial such as “COMPLETED.” The trial metadata 704 includes the Phases field identifying the trial phase such as “PHASE3.” The trial metadata 704 includes the Num. of Enrollment Patients field specifying the total number of patients enrolled such as “408.” The trial metadata 704 further includes the Start Date field indicating when the trial began such as “Feb. 19, 2019” and the Complete Data field indicating when the trial was completed such as “Sep. 20, 2021.”

The structured eligibility criteria 706 provides a detailed representation of the inclusion and exclusion criteria for the clinical trial. The structured eligibility criteria 706 is organized in a tabular format with fields for Component ID, Component Type, Temporal Pattern Category, Event X Type, Event X Concept, Event Y Type, Event Y Concept, and Description. The Component ID field contains a unique identifier for each eligibility criterion component. The Component Type field indicates whether the criterion is an “Inclusion Criterion” or “Exclusion Criterion.” The Temporal Pattern Category field specifies the temporal relationship between clinical events using standardized pattern classifications from the Time Event Ontology such as “XBeforeYwithTime.” The Event X Type field indicates the type of clinical event such as “hasCondition.” The Event X Concept field specifies the standardized clinical concept involved in the criterion such as “Septic Shock.” The Event Y Type field indicates the type of the related clinical event such as “hasObservation.” The Event Y Concept field specifies the related clinical concept such as “Admission to Intensive Care Unit.” The Description field provides a natural language explanation of the complete eligibility criterion including the temporal relationship and clinical events such as “Event X [hasDiagnosis Concept] occurred at least t time units after Event Y [hasObservation Concept].”

In example aspects, the trialist agent 606 generates the trial knowledge graph 700 by applying natural language processing techniques to extract key components from clinical trial protocols retrieved from ClinicalTrials.gov. The trialist agent 606 uses a concept extraction prompt designed based on the Criteria 2 Query 3.0 framework to identify clinical concepts within eligibility criteria text and annotate them into standardized domains including Demographics, Condition, Device, Procedure, Drug, Measurement, Observation, and Visit. The trialist agent 606 further extracts associated values and temporal information related to these clinical concepts. For eligibility criteria containing multiple concepts such as “Allergy to vitamin C, hydrocortisone, or thiamine,” the trialist agent 606 decomposes the text into individual concept annotations. For criteria with omitted concepts such as “Patients<18 years” where the concept “age” is implied but not explicitly stated, the trialist agent 606 infers and annotates the missing concept.

In example aspects, the trialist agent 606 standardizes the extracted clinical concepts using external ontology sources to ensure compatibility with OMOP-CDM schemas. The trialist agent 606 queries the Unified Medical Language System (UMLS) dictionary and Observational Health Data Sciences and Informatics (OHDSI) APIs to map concepts that may be expressed differently across trials to consistent standardized representations. For example, the concepts “ICU” and “Intensive Care Unit” are both mapped to the standardized concept “Admission to Intensive Care Unit.” The trialist agent 606 further maps each standardized concept to its corresponding concept ID within the OMOP-CDM vocabulary including condition codes from ICD-9 and ICD-10, drug codes from RxNorm, measurement codes from LOINC, and procedure codes from SNOMED CT. This standardization process ensures that the structured eligibility criteria 706 contains concept representations that can be directly queried against EHR databases by the informatician agent 608.

In example aspects, the trialist agent 606 identifies and classifies temporal patterns within eligibility criteria using the Time Event Ontology framework. The trialist agent 606 parses temporal modifiers from natural language text including phrases such as “within,” “before,” “after,” “during,” and associated time values. The trialist agent 606 extracts numerical time values and associated units such as “3 months” and normalizes these to standard representations. The trialist agent 606 classifies the temporal relationships between clinical events according to standardized temporal pattern categories such as “X Before Y with Time” indicating that Event X occurred before Event Y with a specified time constraint, “X After Y with Time” indicating that Event X occurred after Event Y with a specified time constraint, or “X During Y” indicating that Event X occurred during the occurrence of Event Y. The Temporal Pattern Category field in the structured eligibility criteria 706 stores these standardized temporal pattern classifications to enable accurate temporal logic implementation by the informatician agent 608 when generating SQL queries with time-based filtering and temporal joins.

In example aspects, the trial metadata 704 provides essential attributes for each trial node in the trial knowledge graph constructed and maintained by the trialist agent 606. Each trial node corresponds to a specific clinical trial and includes the trial metadata 704 as node attributes. The trial metadata 704 enables the trialist agent 606 to efficiently retrieve relevant trials in response to complex queries from the supervisor agent 602 such as “Retrieve all sepsis clinical trials that target hydrocortisone as the intervention but exclude pregnant women, with a sample size greater than 100.” The trialist agent 606 uses the Number of Enrollment Patients field to filter trials based on sample size requirements, the Phases field to filter trials based on development stage, the Study Status field to filter trials based on completion status, and the Start Date and Complete Data fields to filter trials based on temporal windows. This metadata-driven retrieval capability enables the trialist agent 606 to provide the supervisor agent 602 with curated sets of relevant historical trials that can inform the design of new target trial emulations.

In example aspects, the standardized concept mapping 702 illustrates how the trialist agent 606 integrates the structured trial information into the broader trial knowledge graph architecture. The knowledge graph comprises nodes representing clinical trials, trial components, and clinical concepts, with edges representing the relationships between these entities. The Trial-Component relationships connect each trial node to its associated eligibility criteria nodes, treatment strategy nodes, and outcome measure nodes. The Component-Concept relationships connect each trial component node to the specific clinical concept nodes involved in that component. The concept nodes are linked to standardized vocabulary codes from OMOP-CDM including ICD codes for conditions, RxNorm codes for drugs, LOINC codes for measurements, and SNOMED CT codes for procedures. This hierarchical graph structure enables the trialist agent 608 to traverse the knowledge graph to identify all relevant OMOP-CDM concept IDs associated with each eligibility criterion, treatment definition, and outcome measure specified in the target trial protocol.

Thus, the structured trial representation 700 demonstrates the comprehensive standardization process performed by the trialist agent 606 to transform natural language trial protocols into structured, machine-readable representations that are compatible with OMOP-CDM schemas and enable seamless integration with downstream agents in the multi-agent clinical trial emulation system.

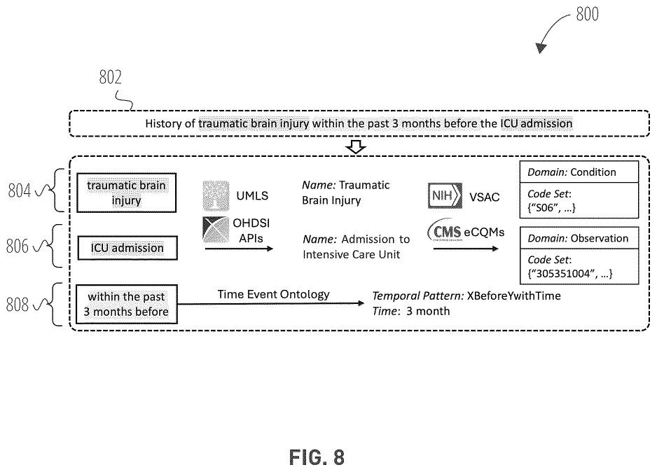

FIG. 8 is a diagram illustrating a criterion parsing diagram 800 showing the process of parsing and standardizing an eligibility criterion according to some examples. The criterion parsing diagram 800 demonstrates how the trialist agent 606 extracts clinical concepts from natural language text and maps them to standardized ontologies compatible with OMOP-CDM schemas.

As shown in the example of FIG. 8, the criterion parsing diagram 800 illustrates the decomposition of a raw eligibility criterion 802 stating “History of traumatic brain injury within the past 3 months before the ICU admission.” The trialist agent 606 parses this natural language text to extract clinical concepts, temporal modifiers, and their relationships, and then standardizes each component through a series of mapping operations.

The criterion parsing diagram 800 includes a first concept mapping pipeline 804 for mapping the first extracted clinical concept to standardized vocabularies. The first concept mapping pipeline 804 begins with the extracted concept “traumatic brain injury” and proceeds through a series of standardization steps. The concept is first queried against the Unified Medical Language System (UMLS) and OHDSI APIs to identify the standardized concept name “Traumatic Brain Injury.” The standardized concept is then mapped through NIH Value Set Authority Center (VSAC) and CMS electronic Clinical Quality Measures (eCQMs) to identify the appropriate standardized code set including ICD codes such as “S06.” The first concept mapping pipeline 804 concludes with domain classification identifying the concept as belonging to the “Condition” domain within the OMOP-CDM framework.

The criterion parsing diagram 800 includes a second concept mapping pipeline 806 for mapping the second extracted clinical concept to standardized vocabularies. The second concept mapping pipeline 806 begins with the extracted concept “ICU admission” and proceeds through the same series of standardization steps as the first concept mapping pipeline 804. The concept is queried against UMLS and OHDSI APIs to identify the standardized concept name “Admission to Intensive Care Unit.” The standardized concept is then mapped through NIH VSAC and CMS eCQMs to identify the appropriate standardized code set including observation codes such as “305351004.” The second concept mapping pipeline 806 concludes with domain classification identifying the concept as belonging to the “Observation” domain within the OMOP-CDM framework.

The criterion parsing diagram 800 includes a temporal pattern mapping pipeline 808 for extracting and standardizing temporal relationships between clinical concepts. The temporal pattern mapping pipeline 808 begins with the extracted temporal phrase “within the past 3 months before” from the raw eligibility criterion 802. The temporal phrase is analyzed using the Time Event Ontology to identify the temporal pattern category as “X Before Y with Time” indicating that Event X (traumatic brain injury) occurred before Event Y (ICU admission) with a specified time constraint. The temporal pattern mapping pipeline 808 further extracts the time value of “3 months” from the natural language text and standardizes this temporal constraint for use in downstream database queries.

In example aspects, the criterion parsing diagram 800 demonstrates how the trialist agent 606 applies natural language processing techniques to parse complex eligibility criteria containing multiple clinical concepts and temporal relationships. The trialist agent 606 identifies individual clinical concepts within the raw eligibility criterion 802 and determines their semantic types including conditions, observations, procedures, drugs, and measurements. The trialist agent 606 maps each identified concept to standardized clinical ontologies through the UMLS dictionary and OHDSI APIs to ensure that identical concepts expressed differently across trials are normalized to consistent representations compatible with OMOP-CDM schemas.

In example aspects, the trialist agent 606 leverages multiple external ontology sources to achieve comprehensive concept standardization. The UMLS provides a unified framework for mapping clinical concepts to standardized names across different terminologies. The NIH VSAC provides access to value sets used in clinical quality measures and research. The OHDSI APIs enable querying of standardized vocabularies within the OMOP-CDM framework including condition codes from ICD-9 and ICD-10, drug codes from RxNorm, measurement codes from LOINC, and procedure codes from SNOMED CT. The CMS eCQMs provide standardized definitions for clinical quality measures used in healthcare reporting. By integrating these multiple ontology sources, the trialist agent 606 ensures that extracted concepts are mapped to the most appropriate standardized codes for downstream use by the informatician agent 608 in generating SQL queries against EHR databases.

In example aspects, the trialist agent 606 applies the Time Event Ontology to identify and classify temporal patterns within eligibility criteria. The Time Event Ontology provides a structured framework for representing complex temporal relationships between clinical events including patterns such as “X Before Y with Time,” “X After Y with Time,” “X During Y,” and other temporal constructs. The trialist agent 606 parses temporal modifiers from natural language text including phrases such as “within,” “before,” “after,” “during,” and associated time values. The trialist agent 606 extracts numerical time values and associated units from the text and normalizes these to standard representations. The standardized temporal patterns enable the informatician agent 608 to generate SQL queries with accurate temporal joins and time-based filtering logic that correctly represent the eligibility criteria when applied to time-stamped EHR data.

In example aspects, the outputs of the first concept mapping pipeline 804, second concept mapping pipeline 806, and temporal pattern mapping pipeline 808 are synthesized to create a structured representation of the complete eligibility criterion. This structured representation includes the standardized concept IDs for each clinical concept, their associated domain classifications, the temporal pattern category, and the temporal constraint value. The structured representation is compatible with OMOP-CDM schemas and provides the necessary information for the informatician agent 608 to generate executable SQL queries. The informatician agent 608 uses the standardized concept IDs to identify relevant records in EHR databases, applies domain classifications to query the appropriate OMOP-CDM tables, and implements the temporal pattern logic to correctly filter patients based on the timing relationships between clinical events. This end-to-end standardization process ensures that eligibility criteria expressed in natural language are accurately translated into database queries that retrieve the correct patient cohorts from real-world data sources.

Thus, the criterion parsing diagram 800 illustrates the comprehensive process by which the trialist agent 606 transforms unstructured natural language eligibility criteria into structured, standardized representations compatible with OMOP-CDM schemas through systematic extraction of clinical concepts, mapping to standardized ontologies, and classification of temporal patterns.

FIG. 9 is a flowchart illustrating a process 900 for performing clinical trial emulation and iteratively refining the design of the emulated trial using multi-agent learning, in accordance with some examples. For explanatory purposes, the process 900 is primarily described herein with reference to the clinical trial design server 118 and the customer client device 102 of FIG. 1. However, one or more blocks (or operations) of the process 900 may be performed by one or more other components, and/or by other suitable devices. Further for explanatory purposes, the blocks (or operations) of the process 900 are described herein as occurring in serial, or linearly. However, multiple blocks (or operations) of the process 900 may occur in parallel or concurrently. In addition, the blocks (or operations) of the process 900 need not be performed in the order shown and/or one or more blocks (or operations) of the process 900 need not be performed and/or can be replaced by other operations. The process 900 may be terminated when its operations are completed. In addition, the process 900 may correspond to a method, a procedure, an algorithm, etc.

The clinical trial design server 118 receives, via a network interface, a target trial specification including trial eligibility criteria, treatments, and outcomes (block 902). At block 904, the clinical trial design server 118 extracts, using a large language model, key components and associated numerical and temporal information from the target trial specification and generating structured and standardized target trial definitions compatible with standardized clinical data formats (e.g., OMOP-CDM schemas). In example embodiments, the large language model is a transformer-based neural network.

In example embodiments, the extracting is performed using a trialist agent including the large language model. The trialist agent is trained via RLHF to generate definitions of eligibility criteria, treatment assignments, and outcome measures and to improve parsing fidelity and compatibility with OMOP-CDM schemas. For example, the multi-agent system includes a trialist agent, an informatician agent, a statistician agent, a clinician agent, and a supervisor agent that coordinates workflow among the agents.

The clinical trial design server 118 generates executable database queries based on the target trial definitions and retrieving patient clinical data from an electronic health record database to produce an analysis-ready dataset stored in memory (block 906). In example embodiments, generating the executable database queries is performed using an informatician agent that retrieves patient clinical data matching the trial eligibility criteria from the electronic health record database. The informatician agent is trained via RLHF to produce the executable database queries.

In example embodiments, the electronic health record database includes real-world data including electronic health records (EHR), medical literature, historical trial results, or any combination thereof.

The clinical trial design server 118 performs data quality assurance, covariate balancing, and survival analysis on the analysis-ready dataset using causal inference methods to estimate treatment effects (block 908). In example embodiments, the performing is performed using a statistician agent that performs selection and explanation of the causal inference methods. The causal inference methods include Propensity Score Matching (PSM), Inverse Probability of Treatment Weighting (IPTW), Cox regression, parametric survival models, random survival forests (RSF), or any combination thereof.

The clinical trial design server 118 obtains feedback for refining clinical parameters and covariate selections (block 910). In example embodiments, obtaining the feedback is performed using a clinician agent that generates clinically grounded suggestions regarding covariate selection, surrogate variable mapping, eligibility relaxation strategies, or any combination thereof.

The clinical trial design server 118 iteratively refines the target trial definitions, analysis-ready dataset, and treatment effect estimates by dynamically communicating said feedback and by optimizing outputs via reinforcement learning with human feedback (RLHF) using a multi-agent system including a plurality of neural network models to generate a trial emulation report including protocol specifications, statistical summaries, and visualizations (block 912). In example embodiments, the clinical trial design server 118 stores the trial emulation report in a database, and transmits the trial emulation report to a client device for display via a clinical trial design client application. The trial emulation report further includes a protocol feasibility assessment.

### Machine Architecture

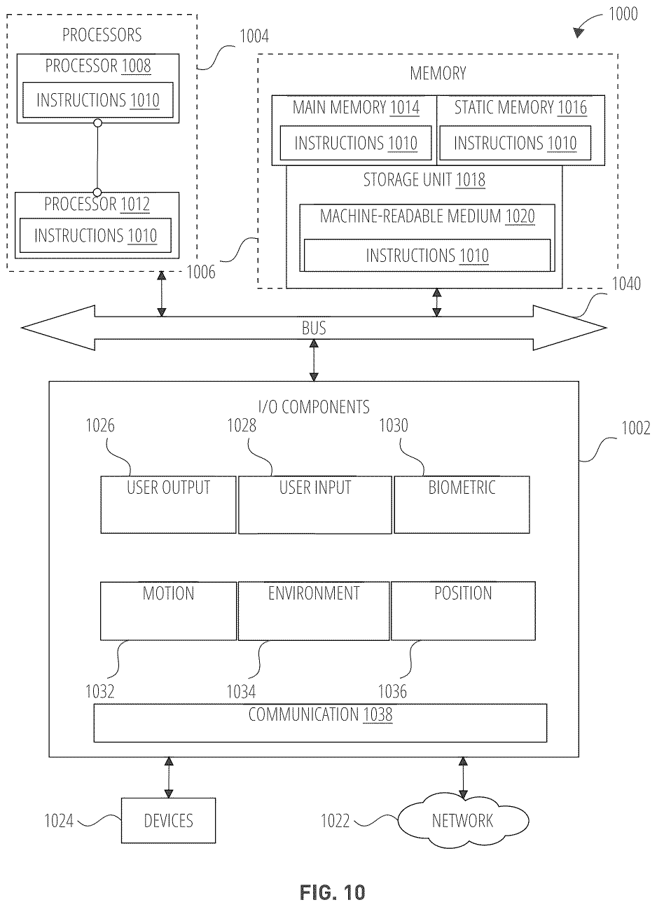

In some examples, components in the architecture 400 can be a machine 1000 as shown in FIG. 10. FIG. 10 is a diagrammatic representation of the machine 1000 within which instructions 1010 (e.g., software, a program, an application, an applet, an application, or other executable code) for causing the machine 1000 to perform any one or more of the methodologies discussed herein may be executed. For example, the instructions 1010 may cause the machine 1000 to execute any one or more of the methods described herein. The instructions 1010 transform the general, non-programmed machine 1000 into a particular machine 1000 programmed to carry out the described and illustrated functions in the manner described. The machine 1000 may operate as a standalone device or may be coupled (e.g., networked) to other machines. In a networked deployment, the machine 1000 may operate in the capacity of a server machine or a client machine in a server-client network environment, or as a peer machine in a peer-to-peer (or distributed) network environment. The machine 1000 may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smartphone, a mobile device, a wearable device (e.g., a smartwatch), a smart home device (e.g., a smart appliance), other smart devices, a web appliance, a network router, a network switch, a network bridge, or any machine capable of executing the instructions 1010, sequentially or otherwise, that specify actions to be taken by the machine 1000. Further, while only a single machine 1000 is illustrated, the term “machine” shall also be taken to include a collection of machines that individually or jointly execute the instructions 1010 to perform any one or more of the methodologies discussed herein. The machine 1000, for example, may comprise the customer client device 102 or any one of a number of server devices forming part of the clinical trial design server 118. In some examples, the machine 1000 may also comprise both client and server systems, with certain operations of a particular method or algorithm being performed on the server-side and with certain operations of the particular method or algorithm being performed on the client-side.

The machine 1000 may include processors 1004, memory 1006, and input/output I/O components 1002, which may be configured to communicate with each other via a bus 1040. In an example, the processors 1004 (e.g., a Central Processing Unit (CPU), a Reduced Instruction Set Computing (RISC) Processor, a Complex Instruction Set Computing (CISC) Processor, a Graphics Processing Unit (GPU), a Digital Signal Processor (DSP), an Application Specific Integrated Circuit (ASIC), a Radio-Frequency Integrated Circuit (RFIC), another processor, or any suitable combination thereof) may include, for example, a processor 1008 and a processor 1012 that execute the instructions 1010. The term “processor” is intended to include multi-core processors that may comprise two or more independent processors (sometimes referred to as “cores”) that may execute instructions contemporaneously. Although FIG. 10 shows multiple processors 1004, the machine 1000 may include a single processor with a single-core, a single processor with multiple cores (e.g., a multi-core processor), multiple processors with a single core, multiple processors with multiples cores, or any combination thereof.

The memory 1006 includes a main memory 1014, a static memory 1016, and a storage unit 1018, both accessible to the processors 1004 via the bus 1040. The main memory 1014, the static memory 1016, and storage unit 1018 store the instructions 1010 embodying any one or more of the methodologies or functions described herein. The instructions 1010 may also reside, completely or partially, within the main memory 1014, within the static memory 1016, within machine-readable medium 1020 within the storage unit 1018, within at least one of the processors 1004 (e.g., within the processor's cache memory), or any suitable combination thereof, during execution thereof by the machine 1000.

The I/O components 1002 may include a wide variety of components to receive input, provide output, produce output, transmit information, exchange information, capture measurements, and so on. The specific I/O components 1002 that are included in a particular machine will depend on the type of machine. For example, portable machines such as mobile phones may include a touch input device or other such input mechanisms, while a headless server machine will likely not include such a touch input device. It will be appreciated that the I/O components 1002 may include many other components that are not shown in FIG. 10. In various examples, the I/O components 1002 may include user output components 1026 and user input components 1028. The user output components 1026 may include visual components (e.g., a display such as a plasma display panel (PDP), a light-emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The user input components 1028 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and force of touches or touch gestures, or other tactile input components), audio input components (e.g., a microphone), and the like.

In further examples, the I/O components 1002 may include biometric components 1030, motion components 1032, environmental components 1034, or position components 1036, among a wide array of other components. For example, the biometric components 1030 include components to detect expressions (e.g., hand expressions, facial expressions, vocal expressions, body gestures, or eye-tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 1032 include acceleration sensor components (e.g., accelerometer), gravitation sensor components, rotation sensor components (e.g., gyroscope).

The environmental components 1034 include, for example, one or cameras (with still image/photograph and video capabilities), illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment.

With respect to cameras, the customer client device 102 may have a camera system comprising, for example, front cameras on a front surface of the customer client device 102 and rear cameras on a rear surface of the customer client device 102. The front cameras may, for example, be used to capture still images and video of a user of the customer client device 102 (e.g., “selfies”). The rear cameras may, for example, be used to capture still images and videos in a more traditional camera mode. In addition to front and rear cameras, the customer client device 102 may also include a 360° camera for capturing 360° photographs and videos.

Further, the camera system of a customer client device 102 may include dual rear cameras (e.g., a primary camera as well as a depth-sensing camera), or even triple, quad or penta rear camera configurations on the front and rear sides of the customer client device 102. These multiple cameras systems may include a wide camera, an ultra-wide camera, a telephoto camera, a macro camera and a depth sensor, for example.

The position components 1036 include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1002 further include communication components 1038 operable to couple the machine 1000 to a network 1022 or devices 1024 via respective coupling or connections. For example, the communication components 1038 may include a network interface component or another suitable device to interface with the network 1022. In further examples, the communication components 1038 may include wired communication components, wireless communication components, cellular communication components, Near Field Communication (NFC) components, Bluetooth° components (e.g., Bluetooth® Low Energy), Wi-Fi® components, and other communication components to provide communication via other modalities. The devices 1024 may be another machine or any of a wide variety of peripheral devices (e.g., a peripheral device coupled via a USB).

Moreover, the communication components 1038 may detect identifiers or include components operable to detect identifiers. For example, the communication components 1038 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 1038, such as location via Internet Protocol (IP) geolocation, location via Wi-Fi® signal triangulation, location via detecting an NFC beacon signal that may indicate a particular location, and so forth.

The various memories (e.g., main memory 1014, static memory 1016, and memory of the processors 1004) and storage unit 1018 may store one or more sets of instructions and data structures (e.g., software) embodying or used by any one or more of the methodologies or functions described herein. These instructions (e.g., the instructions 1010), when executed by processors 1004, cause various operations to implement the disclosed examples.

The instructions 1010 may be transmitted or received over the network 1022, using a transmission medium, via a network interface device (e.g., a network interface component included in the communication components 1038) and using any one of several well-known transfer protocols (e.g., hypertext transfer protocol (HTTP)). Similarly, the instructions 1010 may be transmitted or received using a transmission medium via a coupling (e.g., a peer-to-peer coupling) to the devices 1024.

### Software Architecture

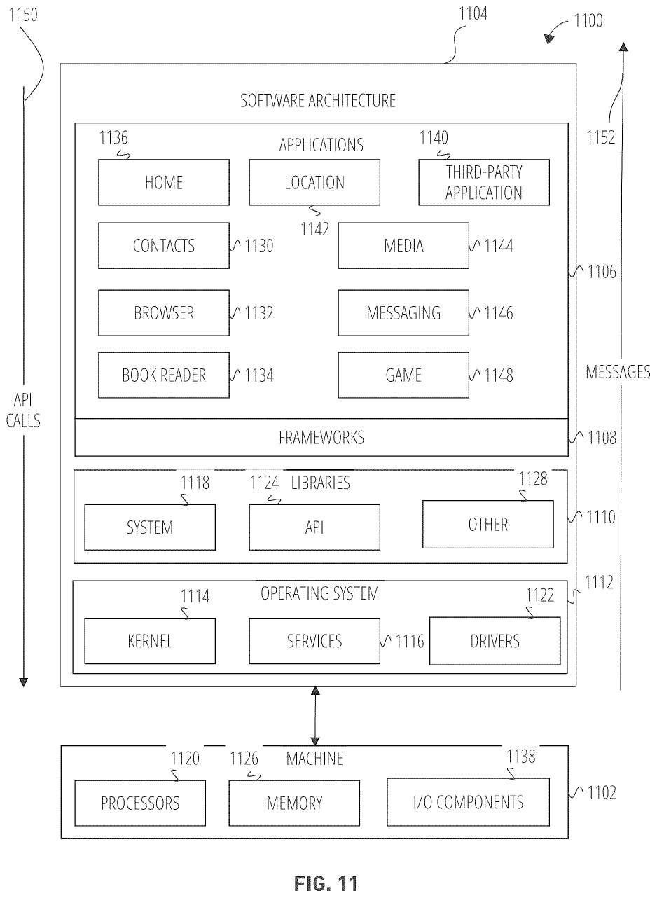

FIG. 11 is a block diagram 1100 illustrating a software architecture 1104, which can be installed on any one or more of the devices described herein. The software architecture 1104 is supported by hardware such as a machine 1102 that includes processors 1120, memory 1126, and I/O components 1138. In this example, the software architecture 1104 can be conceptualized as a stack of layers, where each layer provides a particular functionality. The software architecture 1104 includes layers such as an operating system 1112, libraries 1110, frameworks 1108, and applications 1106. Operationally, the applications 1106 invoke API calls 1150 through the software stack and receive messages 1152 in response to the API calls 1150.

The operating system 1112 manages hardware resources and provides common services. The operating system 1112 includes, for example, a kernel 1114, services 1116, and drivers 1122. The kernel 1114 acts as an abstraction layer between the hardware and the other software layers. For example, the kernel 1114 provides memory management, processor management (e.g., scheduling), component management, networking, and security settings, among other functionalities. The services 1116 can provide other common services for the other software layers. The drivers 1122 are responsible for controlling or interfacing with the underlying hardware. For instance, the drivers 1122 can include display drivers, camera drivers, BLUETOOTH® or BLUETOOTH® Low Energy drivers, flash memory drivers, serial communication drivers (e.g., USB drivers), WI-FI® drivers, audio drivers, power management drivers, and so forth.

The libraries 1110 provide a common low-level infrastructure used by the applications 1106. The libraries 1110 can include system libraries 1118 (e.g., C standard library) that provide functions such as memory allocation functions, string manipulation functions, mathematic functions, and the like. In addition, the libraries 1110 can include API libraries 1124 such as media libraries (e.g., libraries to support presentation and manipulation of various media formats such as Moving Picture Experts Group-4 (MPEG4), Advanced Video Coding (H.264 or AVC), Moving Picture Experts Group Layer-3 (MP3), Advanced Audio Coding (AAC), Adaptive Multi-Rate (AMR) audio codec, Joint Photographic Experts Group (JPEG or JPG), or Portable Network Graphics (PNG)), graphics libraries (e.g., an OpenGL framework used to render in two dimensions (2D) and three dimensions (3D) in a graphic content on a display), database libraries (e.g., SQLite to provide various relational database functions), web libraries (e.g., WebKit to provide web browsing functionality), and the like. The libraries 1110 can also include a wide variety of other libraries 1128 to provide many other APIs to the applications 1106.

The frameworks 1108 provide a common high-level infrastructure that is used by the applications 1106. For example, the frameworks 1108 provide various graphical user interface (GUI) functions, high-level resource management, and high-level location services. The frameworks 1108 can provide a broad spectrum of other APIs that can be used by the applications 1106, some of which may be specific to a particular operating system or platform.

In an example, the applications 1106 may include a home application 1136, a contacts application 1130, a browser application 1132, a book reader application 1134, a location application 1142, a media application 1144, a messaging application 1146, a game application 1148, and a broad assortment of other applications such as a third-party application 1140. The applications 1106 are programs that execute functions defined in the programs. Various programming languages can be employed to create one or more of the applications 1106, structured in a variety of manners, such as object-oriented programming languages (e.g., Objective-C, Java, or C++) or procedural programming languages (e.g., C or assembly language). In a specific example, the third-party application 1140 (e.g., an application developed using the ANDROID™ or IOS™ software development kit (SDK) by an entity other than the vendor of the particular platform) may be mobile software running on a mobile operating system such as IOS™, ANDROID™, WINDOWS® Phone, or another mobile operating system. In this example, the third-party application 1140 can invoke the API calls 1150 provided by the operating system 1112 to facilitate functionality described herein.

### Glossary

As used in this disclosure, phrases of the form “at least one of an A, a B, or a C,” “at least one of A, B, or C,” “at least one of A, B, and C,” and the like, should be interpreted to select at least one from the group that comprises “A, B, and C.” Unless explicitly stated otherwise in connection with a particular instance in this disclosure, this manner of phrasing does not mean “at least one of A, at least one of B, and at least one of C.” As used in this disclosure, the example “at least one of an A, a B, or a C,” would cover any of the following selections: {A}, {B}, {C}, {A, B}, {A, C}, {B, C}, and {A, B, C}.

Unless the context clearly requires otherwise, throughout the description and the claims, the words “comprise,” “comprising,” and the like are to be construed in an inclusive sense, as opposed to an exclusive or exhaustive sense, e.g., in the sense of “including, but not limited to.” [0122] As used herein, the terms “connected,” “coupled,” or any variant thereof means any connection or coupling, either direct or indirect, between two or more elements; the coupling or connection between the elements can be physical, logical, or a combination thereof.

Additionally, the words “herein,” “above,” “below,” and words of similar import, when used in this application, refer to this application as a whole and not to any portions of this application. Where the context permits, words using the singular or plural number may also include the plural or singular number respectively.

The word “or” in reference to a list of two or more items, covers all the following interpretations of the word: any one of the items in the list, all the items in the list, and any combination of the items in the list. Likewise, the term “and/or” in reference to a list of two or more items, covers all the following interpretations of the word: any one of the items in the list, all the items in the list, and any combination of the items in the list.

The various features, operations, or processes described herein may be used independently of one another, or may be combined in various ways. All possible combinations and sub-combinations are intended to fall within the scope of this disclosure. In addition, certain method or process blocks may be omitted in some implementations.

Although some examples, e.g., those depicted in the drawings, include a particular sequence of operations, the sequence may be altered without departing from the scope of the present disclosure. For example, some of the operations depicted may be performed in parallel or in a different sequence that does not materially affect the functions as described in the examples. In other examples, different components of an example device or system that implements an example method may perform functions at substantially the same time or in a specific sequence.

### Example Statements

Example 1 is a computer-implemented method for automated clinical trial design, comprising: receiving, via a network interface, a target trial specification comprising trial eligibility criteria, treatments, and outcomes; extracting, using a large language model, key components and associated numerical and temporal information from the target trial specification and generating structured and standardized target trial definitions compatible with OMOP-CDM schemas; generating executable database queries based on the target trial definitions and retrieving patient clinical data from an electronic health record database to produce an analysis-ready dataset stored in memory; performing data quality assurance, covariate balancing, and survival analysis on the analysis-ready dataset using causal inference methods to estimate treatment effects; obtaining feedback for refining clinical parameters and covariate selections; and iteratively refining the target trial definitions, analysis-ready dataset, and treatment effect estimates by dynamically communicating said feedback and by optimizing outputs via reinforcement learning with human feedback (RLHF) using a multi-agent system comprising a plurality of neural network models to generate a trial emulation report comprising protocol specifications, statistical summaries, and visualizations.
In Example 2, the subject matter of Example 1 includes, storing the trial emulation report in a database; and transmitting the trial emulation report to a client device for display via a clinical trial design client application.
In Example 3, the subject matter of Examples 1-2 includes, wherein the extracting is performed using a trialist agent comprising the large language model.
In Example 4, the subject matter of Example 3 includes, wherein the trialist agent is trained via RLHF to generate definitions of eligibility criteria, treatment assignments, and outcome measures and to improve parsing fidelity and compatibility with common data model schemas such as OMOP and PCORnet.
In Example 5, the subject matter of Examples 1-4 includes, wherein the generating the executable database queries is performed using an informatician agent that retrieves patient clinical data matching the trial eligibility criteria from the electronic health record database.
In Example 6, the subject matter of Example 5 includes, wherein the informatician agent is trained via RLHF to produce the executable database queries.
In Example 7, the subject matter of Examples 1-6 includes, wherein the performing is performed using a statistician agent that performs selection and explanation of the causal inference methods.
In Example 8, the subject matter of Example 7 includes, wherein the causal inference methods include Propensity Score Matching (PSM), Inverse Probability of Treatment Weighting (IPTW), Cox regression, parametric survival models, random survival forests (RSF), or any combination thereof.
In Example 9, the subject matter of Examples 1-8 includes, wherein the obtaining feedback is performed using a clinician agent that generates clinically grounded suggestions regarding covariate selection, surrogate variable mapping, eligibility relaxation strategies, or any combination thereof.
In Example 10, the subject matter of Examples 1-9 includes, wherein the multi-agent system comprises a trialist agent, an informatician agent, a statistician agent, a clinician agent, and a supervisor agent that coordinates workflow among the agents.
In Example 11, the subject matter of Examples 1-10 includes, wherein the electronic health record database comprises real-world data including electronic health records (EHR), medical literature, historical trial results, or any combination thereof.
In Example 12, the subject matter of Examples 1-11 includes, wherein the trial emulation report further comprises a protocol feasibility assessment.
In Example 13, the subject matter of Examples 1-12 includes, wherein the large language model is a transformer-based neural network.
Example 14 is a system comprising: at least one processor; at least one memory component storing instructions that, when executed by the at least one processor, cause the at least one processor to perform operations comprising: receiving, via a network interface, a target trial specification comprising trial eligibility criteria, treatments, and outcomes; extracting, using a large language model, key components and associated numerical and temporal information from the target trial specification and generating structured and standardized target trial definitions compatible with OMOP-CDM schemas; generating executable database queries based on the target trial definitions and retrieving patient clinical data from an electronic health record database to produce an analysis-ready dataset stored in memory; performing data quality assurance, covariate balancing, and survival analysis on the analysis-ready dataset using causal inference methods to estimate treatment effects; obtaining feedback for refining clinical parameters and covariate selections; and iteratively refining the target trial definitions, analysis-ready dataset, and treatment effect estimates by dynamically communicating said feedback and by optimizing outputs via reinforcement learning with human feedback (RLHF) using a multi-agent system comprising a plurality of neural network models to generate a trial emulation report comprising protocol specifications, statistical summaries, and visualizations.
In Example 15, the subject matter of Example 14 includes, the operations further comprising: storing the trial emulation report in a database; and transmitting the trial emulation report to a client device for display via a clinical trial design client application.
In Example 16, the subject matter of Examples 14-15 includes, wherein the extracting is performed using a trialist agent comprising the large language model.
In Example 17, the subject matter of Example 16 includes, wherein the trialist agent is trained via RLHF to generate definitions of eligibility criteria, treatment assignments, and outcome measures and to improve parsing fidelity and compatibility with OMOP-CDM schemas.
In Example 18, the subject matter of Examples 14-17 includes, wherein the generating the executable database queries is performed using an informatician agent that retrieves patient clinical data matching the trial eligibility criteria from the electronic health record database.
Example 19 is a non-transitory computer-readable storage medium storing instructions that, when executed by at least one processor, cause the at least one processor to perform operations comprising: receiving, via a network interface, a target trial specification comprising trial eligibility criteria, treatments, and outcomes; extracting, using a large language model, key components and associated numerical and temporal information from the target trial specification and generating structured and standardized target trial definitions compatible with OMOP-CDM schemas; generating executable database queries based on the target trial definitions and retrieving patient clinical data from an electronic health record database to produce an analysis-ready dataset stored in memory; performing data quality assurance, covariate balancing, and survival analysis on the analysis-ready dataset using causal inference methods to estimate treatment effects; obtaining feedback for refining clinical parameters and covariate selections; and iteratively refining the target trial definitions, analysis-ready dataset, and treatment effect estimates by dynamically communicating said feedback and by optimizing outputs via reinforcement learning with human feedback (RLHF) using a multi-agent system comprising a plurality of neural network models to generate a trial emulation report comprising protocol specifications, statistical summaries, and visualizations.
In Example 20, the subject matter of Example 19 includes, the operations further comprising: storing the trial emulation report in a database; and transmitting the trial emulation report to a client device for display via a clinical trial design client application. “Carrier signal” refers to any intangible medium that is capable of storing, encoding, or carrying instructions for execution by the machine, and includes digital or analog communications signals or other intangible media to facilitate communication of such instructions. Instructions may be transmitted or received over a network using a transmission medium via a network interface device.

“Client device” refers to any machine that interfaces to a communications network to obtain resources from one or more server systems or other client devices. A client device may be, but is not limited to, a mobile phone, desktop computer, laptop, portable digital assistants (PDAs), smartphones, tablets, ultrabooks, netbooks, laptops, multi-processor systems, microprocessor-based or programmable consumer electronics, game consoles, set-top boxes, or any other communication device that a user may use to access a network.

“Communication network” refers to one or more portions of a network that may be an ad hoc network, an intranet, an extranet, a virtual private network (VPN), a local area network (LAN), a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other types of cellular or wireless coupling. In this example, the coupling may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long-range protocols, or other data transfer technology.

“Component” refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various examples, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein. A hardware component may also be implemented mechanically, electronically, or any suitable combination thereof. For example, a hardware component may include dedicated circuitry or logic that is permanently configured to perform certain operations. A hardware component may be a special-purpose processor, such as a field-programmable gate array (FPGA) or an application specific integrated circuit (ASIC). A hardware component may also include programmable logic or circuitry that is temporarily configured by software to perform certain operations. For example, a hardware component may include software executed by a general-purpose processor or other programmable processor. Once configured by such software, hardware components become specific machines (or specific components of a machine) uniquely tailored to perform the configured functions and are no longer general-purpose processors. It will be appreciated that the decision to implement a hardware component mechanically, in dedicated and permanently configured circuitry, or in temporarily configured circuitry (e.g., configured by software), may be driven by cost and time considerations. Accordingly, the phrase “hardware component” (or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering examples in which hardware components are temporarily configured (e.g., programmed), each of the hardware components need not be configured or instantiated at any one instance in time. For example, where a hardware component comprises a general-purpose processor configured by software to become a special-purpose processor, the general-purpose processor may be configured as respectively different special-purpose processors (e.g., comprising different hardware components) at different times. Software accordingly configures a particular processor or processors, for example, to constitute a particular hardware component at one instance of time and to constitute a different hardware component at a different instance of time. Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In examples in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output. Hardware components may also initiate communications with input or output devices, and can operate on a resource (e.g., a collection of information). The various operations of example methods described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions described herein. As used herein, “processor-implemented component” refers to a hardware component implemented using one or more processors. Similarly, the methods described herein may be at least partially processor-implemented, with a particular processor or processors being an example of hardware. For example, at least some of the operations of a method may be performed by one or more processors 1004 or processor-implemented components. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an API). The performance of certain of the operations may be distributed among the processors, not only residing within a single machine, but deployed across a number of machines. In some examples, the processors or processor-implemented components may be located in a single geographic location (e.g., within a home environment, an office environment, or a server farm). In other examples, the processors or processor-implemented components may be distributed across a number of geographic locations.

“Computer-readable storage medium” refers to both machine-storage media and transmission media. Thus, the terms include both storage devices/media and carrier waves/modulated data signals. The terms “machine-readable medium,” “computer-readable medium” and “device-readable medium” mean the same thing and may be used interchangeably in this disclosure.

“Machine storage medium” refers to a single or multiple storage devices and media (e.g., a centralized or distributed database, and associated caches and servers) that store executable instructions, routines and data. The term shall accordingly be taken to include, but not be limited to, solid-state memories, and optical and magnetic media, including memory internal or external to processors. Specific examples of machine-storage media, computer-storage media and device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks The terms “machine-storage medium,” “device-storage medium,” “computer-storage medium” mean the same thing and may be used interchangeably in this disclosure. The terms “machine-storage media,” “computer-storage media,” and “device-storage media” specifically exclude carrier waves, modulated data signals, and other such media, at least some of which are covered under the term “signal medium.”

“Non-transitory computer-readable storage medium” refers to a tangible medium that is capable of storing, encoding, or carrying the instructions for execution by a machine.

“Signal medium” refers to any intangible medium that is capable of storing, encoding, or carrying the instructions for execution by a machine and includes digital or analog communications signals or other intangible media to facilitate communication of software or data. The term “signal medium” shall be taken to include any form of a modulated data signal, carrier wave, and so forth. The term “modulated data signal” means a signal that has one or more of its characteristics set or changed in such a matter as to encode information in the signal. The terms “transmission medium” and “signal medium” mean the same thing and may be used interchangeably in this disclosure.

What is claimed is:

1. A computer-implemented method for automated clinical trial design, comprising:
extracting, using a large language model, key components and associated numerical and temporal information for trials from registries and generating a trial knowledge graph containing structured and standardized trial information compatible with standardized clinical data formats;
receiving, via a network interface, a trial design input comprising at least one of: (i) a natural language clinical research question specifying a disease and an intervention of interest, (ii) a request to emulate an existing clinical trial;
converting, by querying the trial knowledge graph, the trial design input into a target trial specification comprising structured and standardized trial eligibility criteria, treatments, and outcomes;
generating executable database queries based on the target trial definitions and retrieving patient clinical data from an electronic health record database to produce an analysis-ready dataset stored in memory;
performing data quality assurance, covariate balancing, and survival analysis on the analysis-ready dataset using causal inference methods to estimate treatment effects;
obtaining feedback for refining clinical parameters and covariate selections; and
iteratively refining the target trial definitions, analysis-ready dataset, and treatment effect estimates by dynamically communicating said feedback and by optimizing outputs via reinforcement learning with human feedback (RLHF) using a multi-agent system comprising a plurality of neural network models to generate a trial emulation report comprising protocol specifications, statistical summaries, and visualizations.

2. The method of claim 1, further comprising:
storing the trial emulation report in a database; and
transmitting the trial emulation report to a client device for display via a clinical trial design client application.

3. The method of claim 1, wherein the extracting is performed using a trialist agent comprising the large language model.

4. The method of claim 3, wherein the trialist agent is trained via RLHF to generate definitions of eligibility criteria, treatment assignments, and outcome measures and to improve parsing fidelity and compatibility with standardized clinical data formats.

5. The method of claim 1, wherein the generating the executable database queries is performed using an informatician agent that retrieves patient clinical data matching the trial eligibility criteria from the electronic health record database.

6. The method of claim 5, wherein the informatician agent uses a large language model to produce the executable database queries, and wherein the large language model is fine-tuned using RLHF.

7. The method of claim 1, wherein the performing is performed using a statistician agent that performs selection and explanation of the causal inference methods.

8. The method of claim 7, wherein the causal inference methods include Propensity Score Matching (PSM), Inverse Probability of Treatment Weighting (IPTW), doubly robust estimation, Cox regression, parametric survival models, random survival forests (RSF), or any combination thereof.

9. The method of claim 1, wherein the obtaining feedback is performed using a clinician agent that generates clinically grounded suggestions regarding covariate selection, surrogate variable mapping, eligibility relaxation strategies, or any combination thereof.

10. The method of claim 1, wherein the multi-agent system comprises a trialist agent, an informatician agent, a statistician agent, a clinician agent, and a supervisor agent that coordinates workflow among the agents.

11. The method of claim 1, wherein the electronic health record database comprises real-world data including electronic health records (EHR), medical literature, historical trial results, or any combination thereof.

12. The method of claim 1, wherein the trial emulation report further comprises a protocol feasibility assessment.

13. The method of claim 1, wherein the large language model is a transformer-based neural network.

14. The method of claim 1, wherein the standardized clinical data formats comprise observational medical outcomes partnership common data model (OMOP-CDM) schemas.

15. A system comprising:
at least one processor;
at least one memory component storing instructions that, when executed by the at least one processor, cause the at least one processor to perform operations comprising:
extracting, using a large language model, key components and associated numerical and temporal information for trials from registries and generating a trial knowledge graph containing structured and standardized trial information compatible with standardized clinical data formats;
receiving, via a network interface, a trial design input comprising at least one of: (i) a natural language clinical research question specifying a disease and an intervention of interest;
(ii) a request to emulate an existing clinical trial;
converting, by querying the trial knowledge graph, the trial design input into a target trial specification comprising structured and standardized trial eligibility criteria, treatments, and outcomes;
generating executable database queries based on the target trial definitions and retrieving patient clinical data from an electronic health record database to produce an analysis-ready dataset stored in memory;
performing data quality assurance, covariate balancing, and survival analysis on the analysis-ready dataset using causal inference methods to estimate treatment effects;
obtaining feedback for refining clinical parameters and covariate selections; and
iteratively refining the target trial definitions, analysis-ready dataset, and treatment effect estimates by dynamically communicating said feedback and by optimizing outputs via reinforcement learning with human feedback (RLHF) using a multi-agent system comprising a plurality of neural network models to generate a trial emulation report comprising protocol specifications, statistical summaries, and visualizations.

16. The system of claim 15, the operations further comprising:
storing the trial emulation report in a database; and
transmitting the trial emulation report to a client device for display via a clinical trial design client application.

17. The system of claim 15, wherein the extracting is performed using a trialist agent comprising the large language model.

18. The system of claim 17, wherein the trialist agent is trained via RLHF to generate definitions of eligibility criteria, treatment assignments, and outcome measures and to improve parsing fidelity and compatibility with standardized clinical data formats.

19. The system of claim 15, wherein the generating the executable database queries is performed using an informatician agent that retrieves patient clinical data matching the trial eligibility criteria from the electronic health record database.

20. A non-transitory computer-readable storage medium storing instructions that, when executed by at least one processor, cause the at least one processor to perform operations comprising:
extracting, using a large language model, key components and associated numerical and temporal information for trials from registries and generating a trial knowledge graph containing structured and standardized trial information compatible with standardized clinical data formats;
receiving, via a network interface, a trial design input comprising at least one of: (i) a natural language clinical research question specifying a disease and an intervention of interest;
(ii) a request to emulate an existing clinical trial;
converting, by querying the trial knowledge graph, the trial design input into a target trial specification comprising structured and standardized trial eligibility criteria, treatments, and outcomes;
generating executable database queries based on the target trial definitions and retrieving patient clinical data from an electronic health record database to produce an analysis-ready dataset stored in memory;
performing data quality assurance, covariate balancing, and survival analysis on the analysis-ready dataset using causal inference methods to estimate treatment effects;
obtaining feedback for refining clinical parameters and covariate selections; and
iteratively refining the target trial definitions, analysis-ready dataset, and treatment effect estimates by dynamically communicating said feedback and by optimizing outputs via reinforcement learning with human feedback (RLHF) using a multi-agent system comprising a plurality of neural network models to generate a trial emulation report comprising protocol specifications, statistical summaries, and visualizations.
